(19) United States
(12) Patent Application Publication
(10) Pub. No.: US 2006/0204971 A1
(43) Pub. Date: Sep. 14, 2006

(54) Oligonucleotides for multiplexed binding assays

(76) Inventors: Shobha A. Varde, Hillsborough, NJ (US); Mark A. Kunkel, Lawrenceville, NJ (US); Anton Yuryev, Germantown, MD (US)

Correspondence Address: Marc Karish;Sheldon & Mak, 225 S. Lake Avenue, 9th Floor, Pasadena, CA, 91101, US

(21) Appl. No.: 11/078,591

(22) Filed: Mar. 11, 2005

(51) Int. Cl. C12Q 1/68; C12M 1/34

(52) U.S. Cl. 435/6; 435/287.2

(57) ABSTRACT

An assay system for an analyte, the system having a solid support; and six or more pairs of oligonucleotides, each oligonucleotide of the pair being at least partially complementary to the other oligonucleotide of the pair. Each pair has a first oligonucleotide and a second oligonucleotide, the first oligonucleotide being immobilized to the solid support, and the second oligonucleotide having an analyte binding agent attached thereto. The oligonucleotides have a maximum of three consecutive identical nucleotides and less than about 10% cross-hybridization when used in assays conducted at from about 37° C. to about 45° C.

### BACKGROUND

The following description provides a summary of information relevant to the present invention and is not an indication that any of the information provided or publications referenced herein is prior art to the presently claimed invention.

The hybridization of oligonucleotides is useful for a wide variety of analytical and diagnostic assays, including the sorting, detection, and identification of analytes. It is desirable to screen samples for ever greater numbers of elements and characteristics at the same time. Accordingly, the number of oligonucleotides being used in a given assay continues to increase. As the number of oligonucleotides used in a given assay increase, it is important that non-complementary oligonucleotides do not cross-hybridize with one another. Unfortunately, such non-specific cross-hybridization is a common problem and is the primary cause of false positive or false negative signals in oligonucleotide-based assays, in particular where multiple oligonucleotide pairs are employed.

Cross-hybridization of oligonucleotides is in part due to the variability in base-stacking energies among the different nucleotides and nucleotide sequence variations within oligonucleotides. Another set of parameters affecting oligonucleotide cross-hybridization is the hybridization reaction conditions. As the length of oligonucleotides used increases to the point where an individual oligonucleotide can fold back upon itself, self-complementarity becomes a problem. It is also important that the oligonucleotides used are not too homologous to common repeating sequences in the human genome, as oligonucleotides that are too homologous will likely bind to undesirable components of a sample.

Other factors that pose problems to the design and implementation of an assay system utilizing multiple oligonucleotides are the desirability that substantially all of the paired oligonucleotides have substantially the same Tm, that all paired oligonucleotides hybridize at substantially the same rate and within the same time frame, and the desirability that the oligonucleotides hybridize together relatively rapidly.

What is needed is an oligonucleotide-based assay system useful for the detection of multiple analytes that utilizes a set of oligonucleotide pairs having no significant cross-hybridization between different pairs and no consequent false positive or negative signals. A need also exists for an oligonucleotide-based assay system where all paired oligonucleotides hybridize at substantially the same rate, have substantially the same Tm, and where complementary oligonucleotide pairs are able to hybridize together relatively rapidly. Moreover, an improved method for generating sets of oligonucleotides for multiplexed binding assays is needed.

### SUMMARY

Accordingly, the present invention, in an embodiment, is directed to an assay system for an analyte, the system having a solid support; and six or more pairs of oligonucleotides. Each oligonucleotide of one pair is at least partially complementary to the other oligonucleotide of that pair. Each pair has a first oligonucleotide and a second oligonucleotide, the first oligonucleotide having being immobilized to the solid support, and the second oligonucleotide having an analyte binding agent attached thereto. The oligonucleotides have a maximum of three consecutive identical nucleotides and less than about 10% cross-hybridization when used in assays conducted at from about 37° C. to about 45° C. In additional embodiments, the system may have at least 12, 24, 48, 96, or 133 pairs of oligonucleotides. Each oligonucleotide may have at least 15 consecutive nucleotides from a different one of SEQ ID NOS: 1-266. Each oligonucleotide may be a different one of SEQ ID NOS: 1-266.

The present invention is also directed to a set of at least 48 pairs of oligonucleotides for use in a binding assay, each oligonucleotide in the set having a maximum of three consecutive identical nucleotides; and less than about 10% cross-hybridization when used in assays conducted at from about 37° C. to about 45° C.

The present invention is also directed to a method of detecting an analyte in a sample, the method having the steps of: selecting six or more complementary oligonucleotide pairs, each pair having a first oligonucleotide and a second oligonucleotide, the first oligonucleotide being immobilized to a solid support, the second oligonucleotide comprising an analyte binding agent attached thereto, each nucleotide having at least 15 consecutive nucleotides from SEQ ID NO:1 to SEQ ID NO:266. A sample having one or more analytes is provided. The one or more analytes are admixed with the second oligonucleotides under conditions where the analyte binding agents are able to bind to their respective analytes. The analytes and second oligonucleotides are further processed to attach a detectable label onto the second oligonucleotide. The six or more oligonucleotide pairs are admixed under conditions that facilitate the selective hybridization of complementary oligonucleotides to form hybridized oligonucleotides with a bound analyte or label. The bound analyte or label is then detected.

The present invention is also directed to a method for generating a collection of nucleic acid sequences, the method comprising the steps of: generating a plurality of oligonucleotides, each of the oligonucleotides having a predetermined Tm. One of the plurality of oligonucleotides is selected for analysis. The selected oligonucleotide is discarded if the selected oligonucleotide has more than: about 3 consecutive self-complementary nucleotides; about 3 consecutive identical nucleotides; or about 6 consecutive purines. The selected oligonucleotide is compared with any oligonucleotides in the collection and discarded if the selected oligonucleotide has more than 12 consecutive matching nucleotides with any oligonucleotide in the collection; or more than 12 consecutive matching nucleotides with the complement of any oligonucleotide in the collection. If undiscarded, the selected oligonucleotide is added to the collection.

The present invention is also directed to an assay kit for an analyte, the kit having a solid substrate; at least 12 non-complementary oligonucleotides bound to the solid substrate, each oligonucleotide having a Tm of from about 54° C. to about 75° C.; and less than about 10% cross-hybridization when used in assays conducted at from about 37° C. to about 45° C.

### DRAWINGS

These features, aspects and advantages of the present invention will become better understood with regard to the following description, appended claims and accompanying figure where:

FIG. 1 is a flowchart illustrating the steps for a method for generating a set of oligonucleotides according to an embodiment of the present invention.

### SEQUENCE LISTING

Accompanying this application is a single diskette that contains the nucleotide sequences referenced herein; and the information on the diskette is incorporated herein by reference.

### DESCRIPTION

The following discussion describes embodiments of the invention and several variations of these embodiments. This discussion should not be construed, however, as limiting the invention to those particular embodiments. Practitioners skilled in the art will recognize numerous other embodiments as well. In all of the embodiments described herein that are referred to as being preferred or particularly preferred, these embodiments are not essential even though they may be preferred.

Definitions

The terms “nucleic acid” and “polynucleotide” are used herein interchangeably to include naturally occurring or synthesized double stranded deoxyribonucleic acid (hereinafter “DNA”), single stranded DNA, or ribonucleic acid (hereinafter “RNA”).

The term “oligonucleotide” as used herein is a nucleic acid that includes linear oligomers of natural or modified monomers, a modified backbone, or modified linkages, including deoxyribonucleosides, ribonucleosides, anomeric forms thereof, peptide nucleic acids (PNAs), and the like, capable of specifically binding to an oligonucleotide or a polynucleotide target by way of a regular pattern of monomer-to-monomer interactions, such as Watson-Crick type of base pairing, or the like. Usually monomers are linked by phosphodiester bonds or analogs thereof to form oligonucleotides ranging in size from a few monomeric units, e.g. 3-4, to several tens of monomeric units. Analogs of phosphodiester linkages include phosphorothioate, phosphorodithioate, phosphoranilidate, phosphoramidate, and the like. Alternative chemistries, such as phosphorothioate, phosphoramidate, and similar such groups resulting in a non-natural backbone may also be used provided that the hybridization efficiencies of the resulting oligonucleotides is not adversely affected. The oligonucleotides can be synthesized by a number of approaches, e.g. Ozaki et al, Nucleic Acids Research, 20:5205-5214 (1992); Agrawal et al, Nucleic Acids Research, 18:5419-5423 (1990); or the like.

The terms “complementary” or “complementarity” refer to the natural binding of polynucleotides by base pairing. For example, the sequence 5′-AGT-3′ binds to the complementary sequence 3′-TCA-5′. Complementarity between two single-stranded molecules may be “partial” such that only some of the nucleic acids bind and form a duplex, or complementarity may be “complete” such that total complementarity exists between the single stranded molecules. The degree of complementarity between the nucleic acid strands has significant effects on the efficiency and strength of the hybridization between the nucleic acid strands.

A nucleic acid “duplex” is formed by the base pairing of complementary strands of DNA or RNA that form antiparallel complexes in which the 3′-terminal end of one strand is oriented and bound to the 5′-terminal end of the opposing strand. This base pairing also comprehends the pairing of “nucleoside analogs”, such as deoxyinosine, nucleosides with 2-aminopurine bases, and the like, that may be employed. As used herein, “nucleoside” includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties that are capable of specific hybridization, e.g. described by Scheit, Nucleotide Analogs (John. Wiley, New York, 1980); Uhlman and Peyman, Chemical Reviews, 90:543-584 (1990), or the like. Such analogs include synthetic nucleosides designed to enhance binding properties, reduce degeneracy, increase specificity, and the like.

The terms “hybridize” or “hybridization” are intended to include admixing of at least two nucleic acid sequences under conditions such that when at least two complementary nucleic acid sequences are present, they will form a double-stranded structure through base-pairing.

The invention is directed to an assay system for analytes. The assay system comprises six or more pairs of complementary oligonucleotides. Each pair of complementary oligonucleotides comprises a first oligonucleotide and a second oligonucleotide. The first oligonucleotide of an oligonucleotide pair is immobilized to a solid support. An analyte binding agent is attached to the second oligonucleotide of an oligonucleotide pair. In a preferred embodiment, there are different analyte binding agents linked to the second oligonucleotide of each pair, each different analyte binding agent having a specificity for a different analyte in one or more samples.

The oligonucleotides of the invention are selected to have sequences that minimize the cross-hybridization of oligonucleotides from different oligonucleotide pairs. Each pair of complementary oligonucleotides typically comprises the same total number of nucleotides, or a number of nucleotides that differs by less than five oligonucleotides, and more typically differing by less than three oligonucleotides. The guanine and cytosine content, in combination, “GC content” of each oligonucleotide is typically between about 35% and about 65%, and more typically the GC content of each oligonucleotide is between about 40% and about 60%.

The oligonucleotides of the invention include SEQ ID NO: 1-SEQ ID NO: 266, and variations and subportions thereof, where SEQ ID NO: 1 is complementary to SEQ ID NO: 2, SEQ ID NO: 3 is complementary to SEQ ID NO: 4, SEQ ID NO: 5 is complementary to SEQ ID NO: 6, and so on. The oligonucleotides represented by SEQ ID NOS: 1-266 are shown in Table I.

Oligonucleotides comprising subportions of the above sequences are typically selected from the group consisting of the following: sequences having 15 or more consecutive nucleotides from SEQ ID NOS: 1-266; sequences having 16 or more consecutive nucleotides from SEQ ID NOS: 1-266; sequences having 17 or more consecutive nucleotides from SEQ ID NOS: 1-266; sequences having 18 or more consecutive nucleotides from SEQ ID NOS: 1-266; sequences having 19 or more consecutive nucleotides from SEQ ID NOS: 1-266; or sequences according to SEQ ID NOS: 1-266. Consecutive nucleotides are those that are typically linked directly together by phosphodiester bonds in a polynucleotide chain or strand. Subportions of the oligonucleotides in SEQ ID NOS: 1-266 may be desirable, for example, to change the reaction conditions or temperature. Subportions may be selected by taking one or more nucleotides off of the 3′ end or the 5′ end of the oligonucleotides in SEQ ID NOS: 1-266.

Alternatively, oligonucleotides are selected from the group consisting of sequences having one nucleotide variation in any nucleotide of sequences according to SEQ ID NOS: 1-266; oligonucleotides having two or fewer nucleotide variations in any two or fewer nucleotides of sequences according to SEQ ID NOS: 1-266; oligonucleotides having three or fewer nucleotide variations in any three or fewer nucleotides of sequences according to SEQ ID NOS: 1-266; and oligonucleotides having four or fewer nucleotide variations in any four or fewer nucleotides of sequences according to SEQ ID NOS: 1-266.

An assay system according to the present invention alternatively uses one or more oligonucleotide pairs according to SEQ ID NO: 1-266 or variants thereof described herein; six or more oligonucleotide pairs according to SEQ ID NO: 1-266 or variants thereof described herein, twelve or more oligonucleotide pairs according to SEQ ID NO: 1-266 or variants thereof described herein, twenty-four or more oligonucleotide pairs according to SEQ ID NO: 1-266 or variants thereof described herein, forty-eight or more oligonucleotide pairs according to SEQ ID NOS: 1-266 or variants thereof described herein, ninety-six or more oligonucleotide pairs according to SEQ ID NO: 1-266 or variants thereof described herein, and all oligonucleotide pairs according to SEQ ID NO: 1-266 or variants thereof described herein.

In a particular embodiment, the invention is an assay system for an analyte comprising from forty-eight pairs to fifty-two pairs of complementary oligonucleotides. Each pair of complementary oligonucleotides comprises a first oligonucleotide and a second oligonucleotide, wherein the first oligonucleotide is immobilized to a solid support and the second oligonucleotide has an analyte binding agent attached thereto. The oligonucleotides are selected from the group consisting of SEQ ID NOS: 1-266.

For each oligonucleotide selected from SEQ ID NOS: 1-266 as the first oligonucleotide, the respective complementary oligonucleotide in SEQ ID NOS: 1-266 is selected for the second oligonucleotide. Preferably, all of the first nucleotides are selected from odd numbered sequences in SEQ ID NOS: 1-266 or all of the first nucleotides are selected from even numbered sequences in SEQ ID NOS: 1-266. As explained earlier, subportions, and variants of the selected oligonucleotides can be used, rather than the selected oligonucleotide. It will be understood by those skilled in the art, that variable numbers of oligonucleotides, including, the entire set of 266 oligonucleotides in SEQ ID NOS: 1-266 may be used in a given assay.

The ability to assay a given sample for 48 or more analytes simultaneously saves time and money and allows for better analysis of limited samples. An assay for 48 analytes is typically done in the same time as an assay for 12 analytes, thereby increasing throughput by a factor of four. Typically reagent volumes are the same whether the assay is for 12 analytes or for 48; therefore, the 48 analyte assay has a lower reagent cost per determined analyte. Additionally, because the same amount of sample is typically used in an assay for 48 analytes as in much more limited assays, a 48 analyte assay allows for more detailed analysis when only a small amount of sample is available.

In some embodiments, the oligonucleotides further comprise one or more linker molecules or linker nucleic acid sequences covalently linked to the oligonucleotides having sequences according to SEQ ID NOS: 1-266, or subportions thereof. The linker molecules or linker nucleic acid sequences are typically at the 5′-end or the 3′-end of the oligonucleotides, thus extending beyond the region of the oligonucleotide that is base-paired with the other oligonucleotide of the pair. The linker molecule or linker nucleic acid sequence, when present, typically links and attaches a first oligonucleotide of a pair to an analyte binding agent or links a second oligonucleotide of a pair to a solid support. The linker molecule or linker nucleic acid sequence can also be a part of an oligonucleotide complex generated by sequential hybridization to different oligonucleotide strands, of which one of them is the analyte binding agent, via complementary regions in these oligonucleotides.

Techniques for attaching oligonucleotides to various solid supports are well known in the art, such as described in Matson, R., et al; Biopolymer Synthesis on Polypropylene Supports: Oligonucleotide Arrays; Analytical Biochemistry 224, 110-116 (1995); Milton, R., U.S. Pat. No. 6,146,833; Hermanson, et al., “Immobilized Affinity Ligand Techniques,” pages 64-67 (1992 Academic Press Inc.). Techniques for attaching oligonucleotides to proteins and antibodies are well known in the art, such as described in Reddy et al., U.S. Pat. No. 5,648,213. Other specific oligonucleotide attachment chemistries are described in U.S. Pat. Nos. 6,136,962 and 6,387,626 to Shi et al., where an unmodified nucleic acid molecule is attached to a solid support with a silane. The contents of all of these documents are hereby incorporate herein by reference.

Hybridization reaction conditions are preferably preselected such that each oligonucleotide hybridizes only to its complementary paired oligonucleotide in a mixture of oligonucleotides from the assay system. As referred to herein, hybridizing only to its complementary paired oligonucleotide means no cross-hybridization greater than about 10%. It is preferable that under optimal reaction conditions designed for this set of oligonucleotides, different pairs exhibit no cross-hybridization greater than about 5%, more preferably no greater than about 3%, and even more preferably no greater than about 1%. For example, optimal conditions may include incubation from about 1 to about 2 hours at temperatures in a range of from about 37° C. to about 45° C. in a reaction mixture comprising 850 mM NaCl in MES-buffer. Under these conditions, the oligonucleotide pairs of the invention hybridize together with no cross-hybridization greater than about 3%, and preferably no cross-hybridization greater than about 1%.

Cross-hybridization between oligonucleotide pairs is in part minimized by selecting nucleotide sequences where oligonucleotides from different pairs have a very low degree of complementarity. Further, it is preferable that oligonucleotides from different pairs have a very low amount of complementarity when the alignment of one of the oligonucleotides is shifted relative to the other, including the introduction of a gap or the formation of a loop containing at least some nucleotides that do not undergo base-pairing with the paired oligonucleotide.

Cross-hybridization is tested in an assay system using a single fluorescently labeled second oligonucleotide selected from SEQ ID NOS: 1-266, and a plurality of different first oligonucleotides selected from SEQ ID NOS: 1-266 attached to a solid support. One of the plurality of different first oligonucleotides selected from SEQ ID NOS: 1-266 is complementary to the selected second oligonucleotide. An assay is performed using a relatively high concentration of a single analyte (a concentration near the maximum measurable amount for that assay system), and cross-hybridization is detected as a signal from any non-complementary first oligonucleotide position in the assay. Cross-hybridization is quantified by estimating the amount of fluorescence detected on the solid support where complementary and non-complementary first oligonucleotides were attached, expressed as a percentage of the amount of fluorescence detected from the first oligonucleotide complementary to the fluorescently labeled second oligonucleotide. Each second oligonucleotide was tested individually. A set of the desired number of oligonucleotide pairs can be chosen for a given level of multiplexity and particular hybridization conditions.

Method for Generating Oligonucleotides

The invention further includes a method of generating nucleic acid sequences for complementary oligonucleotide pairs suitable for use in the assay system. An initial step is determining the desired length of the oligonucleotides. The length will typically be determined by the reaction conditions and the number of different oligonucleotides needed for a specific assay. For example, the desired oligonucleotides may have from about 12 to about 24 nucleotides, more preferably from about 16 to about 22 nucleotides, and even more preferably from about 18 to about 20 nucleotides. The oligonucleotides of SEQ ID NOS: 1-266 all have 20 nucleotides. Alternatively all permutations of an oligonucleotide having a preselected length can be considered, and if necessary, another nucleotide added and all the permutations considered again, with the process repeating until a set meeting the desired criteria has been created.

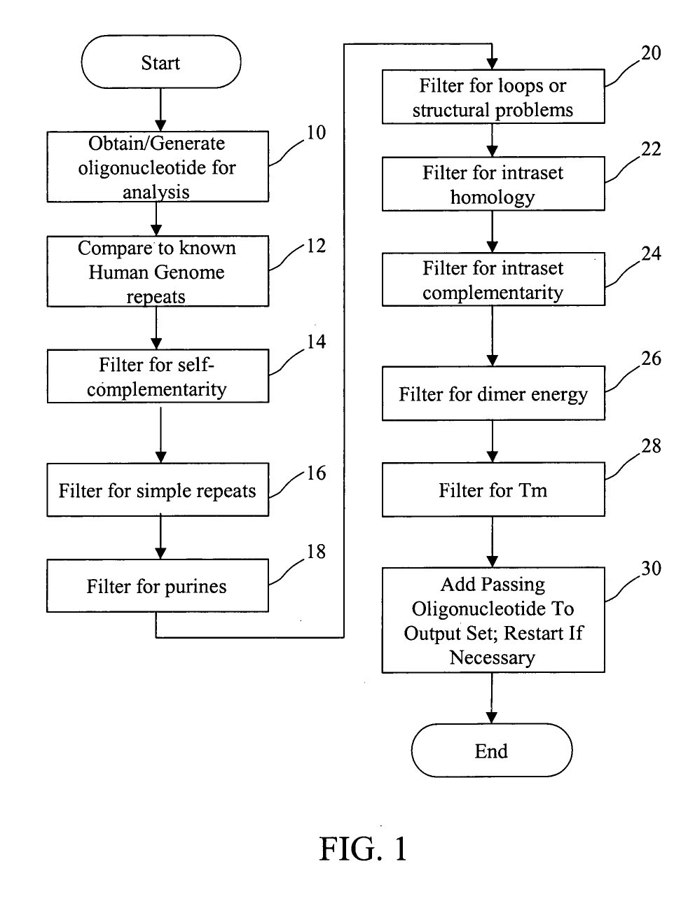

FIG. 1 is a flowchart showing steps for generating a set of oligonucleotides according to an embodiment of the present invention. As shown in box 10, an oligonucleotide is obtained or created for analysis. The oligonucleotide may be obtained from a file of oligonucleotides to be analyzed. Alternatively, the oligonucleotide may be generated systematically by adding single nucleotides to the 3′ end of an oligonucleotide to form an analysis set, the resulting oligonucleotides of the analysis set are then evaluated.

As shown in box 12, an oligonucleotide is compared against repeating sequences known to exist throughout the human genome. Open source tables of such repeating sequences are available from multiple sources, such as, Ensemble at http://www.ensembl.org/. Each oligonucleotide is analyzed by considering a portion of the oligonucleotide at a time by sliding a “window” of a predetermined number of bases along the oligonucleotide and analyzing the nucleotides in the window. In an embodiment, the “window” is eight bases in length. Oligonucleotides shorter in length than the window are not compared against repeating sequences known to exist throughout the human genome.

A “human genome repeat” (HG) score is assigned to each oligonucleotide sequence. Oligonucleotides are given a score related to the frequency that the sequence in a given window appears in the repeat table. The window is moved along the oligonucleotide one base at a time until the length of the oligonucleotide is analyzed. In an embodiment, the number of matches is summed as the window moves along the length of the oligonucleotide. Optionally, the score may be weighted based on proximity of a human genome repeat to the 3′ end of the oligonucleotide. Optionally, oligonucleotides with too many matches to known repeating sequences may be eliminated.

As shown in box 14, the oligonucleotides are analyzed for self recognition using a consecutive self-complementarity filter. An oligonucleotide is compared to its reverse complement to detect consecutive base matches. If the number of consecutive matches exceeds a predetermined number, then the oligonucleotide is eliminated from the set. For example, oligonucleotides with from about 3 to about 8 consecutive self-complementary nucleotides may be eliminated. The oligonucleotides of SEQ ID NOS: 1-266 have no more than 3 consecutive self-complementary nucleotides. An oligonucleotide that is too short to loop back upon itself, or hybridize to itself to form a dimer, is not filtered for self recognition.

As shown in box 16, the oligonucleotides are filtered for simple repeats. Oligonucleotides having more than a predetermined number of consecutive identical nucleotides are eliminated. For example, oligonucleotides with more than two consecutive identical nucleotides can be discarded. The oligonucleotides of SEQ ID NOS: 1-266 all have a maximum of three consecutive identical nucleotides. Optionally, the oligonucleotides are filtered to remove oligonucleotides having sequences of a predetermined length that repeat more than a predetermined number of times. For example, on oligonucleotide having a sequence of six or more nucleotides that repeats more than once can be removed. This type of filtering may be desirable to prevent a complementary oligonucleotide from hybridizing at the wrong position.

As shown in box 18, the oligonucleotides are filtered based on consecutive purines. Consecutive purines affect the melting temperature of bound oligonucleotides. Each oligonucleotide is analyzed to determine the number of consecutive purine bases in an oligonucleotide's primary structure. Oligonucleotides having more than a predetermined number of consecutive purines can be eliminated. The oligonucleotides of SEQ ID NOS: 1-266 have six or less consecutive purines.

As shown in box 20, the oligonucleotides are also filtered for stability of loop formation by calculating the Gibbs free energy calculation (ΔG) for each oligonucleotide. Information on calculating Gibbs free energy can be found in, for example, Breslauer et al., “Predicting DNA duplex stability from the base sequence” Proc. Natl. Acad. Sci. USA 83, 3746-3750 (1986); Freier et al., “Improved free-energy parameters for predictions of RNA duplex stability,” Proc. Natl. Acad. Sci. USA 83, 9373-9377 (1986); Sugimoto, N. (1993) “Relationship between Structure and Function of Nucleic Acids: The Study Using Nearest Neighbor Parameters” Konan University internal publication (Kobe, Japan) 33, 61-67 (1993); SantaLucia J Jr, Hicks D. “The Thermodynamics of DNA Structural Motifs.” Annu. Rev. Biophys. Biomol. Struct. 33, 415-40 (2004); and SantaLucia J Jr, et al., “Effects of GA mismatches on the structure and thermodynamics of RNA internal loops.” Biochemistry. 29, 8813-9 (1990), the entire contents of all of which are incorporated herein in their entirety

Optionally, a computer algorithm, such as Oligo 6.0 available from Molecular Biology Insight, 8685 US Highway 24, Cascade, Colo. 80809-1333, or Lasergene 6.0 available from DNASTAR, Inc, 1228 S. Park St., Madison, Wis. 53715 can be used to analyze each retained sequence for potentially problematic secondary structure or self-complementarity. Oligonucleotides with significant predicted secondary structure or self-complementary structure are eliminated. Each of the oligonucleotides of SEQ ID NOS: 1-266 have a loop ΔG energy of at least about −1.3 kcal/mol.

Each analyzed oligonucleotide that passes the above criteria and has the desired length is then compared to any analyzed oligonucleotides in an output set. Where the oligonucleotides are being generated systematically, and the length of an analyzed oligonucleotide is less than the desired length, then the oligonucleotide is returned to the analysis set.

As shown in box 22, the oligonucleotide is filtered for intraset homology. The oligonucleotide is compared to the other oligonucleotides in the output set for consecutive nucleotide matches in the 5′ to 3′ direction. For example, if an oligonucleotide being analyzed has more than 6 consecutive matching nucleotides with another oligonucleotide in the output set, then the oligonucleotide may be eliminated. No oligonucleotide of SEQ ID NOS: 1-266 has more than 12 consecutive matching nucleotides with any other oligonucleotide of SEQ ID NOS: 1-266.

As shown in box 24, the oligonucleotide is filtered for intraset complementarity. The oligonucleotide is compared to the reverse complements of the other oligonucleotides in the output set for consecutive nucleotide matches. For example, if the oligonucleotide has more than 6 consecutive matching nucleotides with the complement of any other oligonucleotide, then the oligonucleotide may be eliminated. No oligonucleotide of SEQ ID NOS: 1-266 has more than 12 consecutive matching nucleotides with the complement of any other oligonucleotide of SEQ ID NOS: 1-266.

As shown in box 26, the oligonucleotide is filtered based on whether the oligonucleotide is likely to bind to itself or another oligonucleotide in the set as shown by evaluation of the most stable oligonucleotide to oligonucleotide interactions formable by the oligonucleotide. Stability of each of these oligonucleotide to oligonucleotide interactions are determined by Gibbs free energy calculation (ΔG) for each oligonucleotide. Optionally, a computer algorithm, such as Oligo 6.0 available from Molecular Biology Insight, 8685 US Highway 24, Cascade, Colo. 80809-1333, or Lasergene 6.0 available from DNASTAR, Inc, 1228 S. Park St., Madison, Wis. 53715, can be used for these calculations. If the most stable oligonucleotide to oligonucleotide interaction is below a predetermined minimum dimer energy parameter, then the oligonucleotide is eliminated. Typically, the minimum dimer energy parameter is determined based upon the number of bases in the oligonucleotide and the reaction conditions of the assay that the oligonucleotides will be used in. Each of the oligonucleotides of SEQ ID NOS: 1-266 have a dimer ΔG energy of at least about −10.4 kcal/mol. at 1M salt, and 42° C.

As shown in Box 28, each oligonucleotide is passed through a filter to remove oligonucleotides that have undesirable characteristics for the assay they are to be used with. The Tm of the oligonucleotide is evaluated to ensure that the Tm is within a predetermined range for the assay the oligonucleotide is to be used with. Optionally, a computer algorithm, such as Oligo 6.0 available from Molecular Biology Insight, 8685 US Highway 24, Cascade, Colo. 80809-1333, or Lasergene 6.0 available from DNASTAR, Inc, 1228 S. Park St., Madison, Wis. 53715, can be used for these calculations. Oligonucleotides with a Tm outside of the predetermined range are eliminated. The oligonucleotides of SEQ ID NOS: 1-266 all have a Tm from about 54° C. to about 75° C. at 1M salt.

As shown in box 30, an oligonucleotide that passes all of the above filters is added to the output set. Once a new oligonucleotide is added to the output set, the total number of oligonucleotides in the output set is checked to see if the set has reached the desired number of oligonucleotides. If the set is still not large enough, then another set of potential oligonucleotides is analyzed. The process continues until the output set has reached the desired number of oligonucleotides. For example, the process may be repeated until an output set of at least 12, 24, 48, 96, 192, 266 or more oligonucleotides has been created.

Optionally, once the output set has reached the desired number of oligonucleotides, each oligonucleotide in the output set is characterized relative to the other oligonucleotides in the set. Each oligonucleotide may be compared to each other oligonucleotide in the output set for homology to generate a homology score. In an embodiment, the score is a calculation based on the number of identical nucleotides in identical positions of the closest matching oligonucleotide of the output set as a percentage of the total number of nucleotides in the oligonucleotide. Optionally, oligonucleotides may be filtered based on homology as well. For example, oligonucleotides with over about 60% homology to any other oligonucleotide may be eliminated.

Each oligonucleotide may be compared to the reverse complements of the other oligonucleotides in the output set in the same way to generate a complementarity score. Optionally, oligonucleotides may be filtered based on homology as well. For example, oligonucleotides with over about 60% homology to the reverse complement of any other oligonucleotide may be eliminated.

Optionally, each oligonucleotide sequence in the output set, along with its respective human genome repeat score, homology score, complementarity score, and Tm are output to a file or a database for review by a user. Optionally, the oligonucleotides in the output set can be sorted based on one or more of their human genome repeat scores, homology scores, complementarity scores, and Tm's.

Those skilled in the art will recognize that the steps performed in boxes 12 to 28 can be done in different sequence, the sequence presented herein being for illustration only. Preferably, the method of generating oligonucleotides described above is computerized and may be implemented as a method, apparatus, system, or article of manufacture using standard programming and/or engineering techniques to produce software, firmware, hardware, or any combination thereof. The term “article of manufacture” as used herein is intended to encompass a computer program accessible from any computer-readable device, carrier, or media, including the Internet.

Applications

In one possible application of the present invention, the oligonucleotides of SEQ ID NOS: 1-266 are used in a system for detecting Single Nucleotide Polymorphisms (SNP). In an embodiment, at least 48 different first oligonucleotides are selected from SEQ ID NOS: 1-266 and immobilized at the bottom of an assay microtiter well via attachment chemistry. Second oligonucleotides complementary to the first oligonucleotides are also selected from SEQ ID NOS: 1-266. The second oligonucleotides are part of a longer oligonucleotide that functions as an extension primer for a single base extension reaction. The other part of the extension primer is specific to the Single Nucleotide Polymorphism (SNP) being assayed and contains sequences adjacent to the single nucleotide polymorphism.

The assay consists of amplifying a genomic DNA region containing a single nucleotide polymorphism, hybridizing the extension primer to the resulting amplicon, then performing an extension reaction with fluorescently labeled extension terminators representing alternative bases of the single nucleotide polymorphism. The tagged extension produced is then added to an assay microtiter well and allowed to hybridize to the first oligonucleotides. The well is washed and read in a fluorescence reader. The fluorescence read allows detection of the SNP genotype.

An exemplary system for determining the presence or absence of single nucleotide polymorphisms that can be used with the oligonucleotides of SEQ ID NOS: 1-266 is taught in U.S. Pat. No. 6,720,143, the entire contents of which are hereby incorporated herein by reference. The solid support can also be particles or beads that can be optically defined by size and fluorescence using a reader such as a flow cytometer. Those skilled in the art will recognize that the oligonucleotides of SEQ ID NOS 1-266 can be used in other SNP assays, such as those marketed by Third Wave™ Technologies, Inc.; http://www.twt.com.

The analyte binding agent typically comprises an oligonucleotide strand, protein or polypeptide that is capable of specifically binding with an analyte of interest. Preferably, the analyte binding agent is an oligonucleotide strand or oligonucleotide complex generated by sequential hybridization to different oligonucleotide strands, one of which is the analyte binding agent, via complementary regions in the oligonucleotides. Other examples of analyte binding agents are a monoclonal or polyclonal antibody to the analyte, or antigen-binding fragments thereof (e.g., Fab′, F(ab′)₂). Antibodies or antigen-binding fragments thereof is further understood to include chimeric, humanized, recombinant, and other such forms of antibodies. The analyte binding agent encompasses analogues and variants of various immunoreactants (for example, those generated using recombinant DNA techniques) which specifically bind to the target analyte.

Also contemplated as within the scope of the invention are the formation of specific binding pairs comparable to the binding of an antibody to an antigen that can be implemented through the use of other specific protein-based, peptide-based, nucleic acid-based, carbohydrate-based, lipid-based or cell-based binding systems, such as for example a receptor protein or fragment thereof and an analyte ligand, polynucleotide and polynucleotide binding pairs, polynucleotide and protein binding pairs, lipid and protein binding pairs, enzyme and enzyme binding pairs, enzyme and substrate binding pairs, enzyme and inhibitor binding pairs, enzyme and metabolite binding pairs, carbohydrate and protein binding pairs, carbohydrate and lectin binding pairs, protein and drug binding pairs, antibody and cell receptor binding pairs and the like. Further contemplated as within the scope of the invention are chemical-based molecular receptor systems that selectively bind an analyte of interest.

In some embodiments, the solid support comprises a planar surface in which each of the different oligonucleotides is attached to a different predefined region of the substantially planar surface. The solid support typically comprises microarray substrates including but not limited to: flat microscope slides; flexible membranes made of nitrocellulose, nylon, and PVDF; and microwell plates made of glass, polystyrene, polypropylenc, and polycarbonate.

In alternative embodiments, the solid support is a bead, particle, micro particle, magnetic particles or the like, and the terms bead, particle, and micro particle are used interchangeably herein. A typical bead is composed of polystyrene, and may contain other polymeric material. The surface of the particles may contain active chemical groups. The beads typically have diameters in the range of from about 0.04 to about 100 micrometers. The particles may also have ferromagnetic properties. In one embodiment of the present invention, the beads are in a size range from about 1 to about 20 micrometers. The materials and methods that are used to prepare the beads are well known in the art. The particles can be characterized by fluorescent dye attached onto the surface of the particle by standard surface chemistries via biomolecule bridges such as biotin-streptavidin, oligonucleotide, proteins or peptides after particle casting. Particles can also be labeled using a swelling/shrinking process in the presence of the desired fluorophore. Fluorescent particles, such as quantum dots, is an alternative to standard fluorescent dye and can also be attached onto the surface of particles via ligand bridges as known to those skilled in the art. Fluorophores and fluorescent dyes that can be used as detection moieties are well known in the art.

Analytes may be detected by direct or competitive assays. Detection methods may include binding of a single labeled oligonucleotide or sequential hybridizations to capture a labeled single-stranded nucleic acid in the reaction solution onto the solid support. Other detection methods may include the use of a secondary antibody, also called a detection antibody, which has a detection moiety attached thereto. Additional suitable detection moieties include, for example, substrates for enzymatic-based detection, radioactive labels, and the like. Alternative methods of detecting bound analytes are envisioned to be within the scope of the invention, such as for example, but not limited to, direct and competitive immunoassays, ThirdWave SNP detection method, gene expression assays and cell-based assays. Typically assays are conducted at temperatures ranging from about 20° C. to about 55° C.

Typically, the analyte detected by the assay system is selected from the group consisting of genomic DNA, cDNA, mRNA, polypeptides or proteins, carbohydrates, ligands, nucleic acids, lipids, including but not limited to antibodies and binding fragments thereof, hormones, lectins, receptors, steroids, cell-surface antigens, cytokines, viral antigens, bacterial antigens, and drugs of abuse. In a preferred embodiment, the assay system is provided in the form of a kit for the detection of one or more specific analyte.

In alternative embodiments, the analyte can be a cell surface marker. The same overall experimental design for immunochemistry sandwich assay can be modified to detect the presence of a certain cell type or cells expressing a certain cell surface marker by capturing the cells of interest using antibodies as is known to those of skill in the art.

TABLE I

| | |
|---|---|
| SEQ ID NO 1: | 5′-ACGTGATGGTAATAGTGCTG-3′ |
| SEQ ID NO 2: | 5′-CAGCACTATTACCATCACGT-3′ |
| SEQ ID NO 3: | 5′-ACGTGGATTCGTAGTCTGAG-3′ |
| SEQ ID NO 4: | 5′-CTCAGACTACGAATCCACGT-3′ |
| SEQ ID NO 5: | 5′-AGATAGTGAATGGATGGCTG-3′ |
| SEQ ID NO 6: | 5′-CAGCCATCCATTCACTATCT-3′ |
| SEQ ID NO 7: | 5′-AGGTCGTAGGTGCGTTAGAT-3′ |
| SEQ ID NO 8: | 5′-ATCTAACGCACCTACGACCT-3′ |
| SEQ ID NO 9: | 5′-AGTCTGCGGATTACTTGTTG-3′ |
| SEQ ID NO 10: | 5′-CAACAAGTAATCCGCAGACT-3′ |
| SEQ ID NO 11: | 5′-ATGGAGTGCTGTTGATTCTT-3′ |
| SEQ ID NO 12: | 5′-AAGAATCAACAGCACTCCAT-3′ |
| SEQ ID NO 13: | 5′-ATGGTACTTGCGTGAGTTGT-3′ |
| SEQ ID NO 14: | 5′-ACAACTCACGCAAGTACCAT-3′ |
| SEQ ID NO 15: | 5′-ATGTCTAGTGCGTGCTTGTA-3′ |
| SEQ ID NO 16: | 5′-TACAAGCACGCACTAGACAT-3′ |
| SEQ ID NO 17: | 5′-CAGTGCTTGCGTAGAAGTCT-3′ |
| SEQ ID NO 18: | 5′-AGACTTCTACGCAAGCACTG-3′ |
| SEQ ID NO 19: | 5′-CGGTAGGATGGATTATGGAT-3′ |
| SEQ ID NO 20: | 5′-ATCCATAATCCATCCTACCG-3′ |
| SEQ ID NO 21: | 5′-CGTCTAGGTCTTACTGGCGG-3′ |
| SEQ ID NO 22: | 5′-CCGCCAGTAAGACCTAGACG-3′ |
| SEQ ID NO 23: | 5′-CGTCTGTTGTCTATTCTGGA-3′ |
| SEQ ID NO 24: | 5′-TCCAGAATAGACAACAGACG-3′ |
| SEQ ID NO 25: | 5′-CTGCTCGGTATGTTGCTGTT-3′ |
| SEQ ID NO 26: | 5′-AACAGCAACATACCGAGCAG-3′ |
| SEQ ID NO 27: | 5′-CTGGTCTAGGTGGTCTTGCT-3′ |
| SEQ ID NO 28: | 5′-AGCAAGACCACCTAGACCAG-3′ |
| SEQ ID NO 29: | 5′-CTGTTGTTGCGTGCTGTTAT-3′ |
| SEQ ID NO 30: | 5′-ATAACAGCACGCAACAACAG-3′ |
| SEQ ID NO 31: | 5′-CTTACATGGTGAGGATCTGG-3′ |
| SEQ ID NO 32: | 5′-CCAGATCCTCACCATGTAAG-3′ |
| SEQ ID NO 33: | 5′-CTTGCTGGCTCGTATTGTTG-3′ |
| SEQ ID NO 34: | 5′-CAACAATACGAGCCAGCAAG-3′ |
| SEQ ID NO 35: | 5′-GAGCTTGTTGCTTCTTGCAT-3′ |
| SEQ ID NO 36: | 5′-ATGCAAGAAGCAACAAGCTC-3′ |
| SEQ ID NO 37: | 5′-GAGGAGTGCGTCTGTATGTT-3′ |
| SEQ ID NO 38: | 5′-AACATACAGACGCACTCCTC-3′ |
| SEQ ID NO 39: | 5′-GAGGTGGAGGTCTTGATAGG-3′ |
| SEQ ID NO 40: | 5′-CCTATCAAGACCTCCACCTC-3′ |
| SEQ ID NO 41: | 5′-GCTGTTCGTATGTTGATTGT-3′ |
| SEQ ID NO 42: | 5′-ACAATCAACATACGAACAGC-3′ |
| SEQ ID NO 43: | 5′-GGATTGTTCGGTCTTGAAGT-3′ |
| SEQ ID NO 44: | 5′-ACTTCAAGACCGAACAATCC-3′ |
| SEQ ID NO 45: | 5′-GGCTGTAGCGTGGTAGGTAT-3′ |
| SEQ ID NO 46: | 5′-ATACCTACCACGCTACAGCC-3′ |
| SEQ ID NO 47: | 5′-GGTAGTTGTTCGTTGTCTGC-3′ |
| SEQ ID NO 48: | 5′-GCAGACAACGAACAACTACC-3′ |
| SEQ ID NO 49: | 5′-GGTGGTGATGTAGATGGTAA-3′ |
| SEQ ID NO 50: | 5′-TTACCATCTACATCACCACC-3′ |
| SEQ ID NO 51: | 5′-GTATGAGTTGCGTGGATGTT-3′ |
| SEQ ID NO 52: | 5′-AACATCCACGCAACTCATAC-3′ |
| SEQ ID NO 53: | 5′-GTATTCGTGGAGTTGAGTGG-3′ |
| SEQ ID NO 54: | 5′-CCACTCAACTCCACGAATAC-3′ |
| SEQ ID NO 55: | 5′-GTCTTGTCGTCGGTAGTTGT-3′ |
| SEQ ID NO 56: | 5′-ACAACTACCGACGACAAGAC-3′ |
| SEQ ID NO 57: | 5′-GTGAGTGCGGTATGTTGTCT-3′ |
| SEQ ID NO 58: | 5′-AGACAACATACCGCACTCAC-3′ |
| SEQ ID NO 59: | 5′-GTGATGTCTGTTGATGGATC-3′ |
| SEQ ID NO 60: | 5′-GATCCATCAACAGACATCAC-3′ |
| SEQ ID NO 61: | 5′-GTGCTGGTCTTGTTATGTTG-3′ |
| SEQ ID NO 62: | 5′-CAACATAACAAGACCAGCAC-3′ |
| SEQ ID NO 63: | 5′-GTTACTGCGTATGGATTGCG-3′ |
| SEQ ID NO 64: | 5′-CGCAATCCATACGCAGTAAC-3′ |
| SEQ ID NO 65: | 5′-GTTGAGCGGTCTTATGTTGC-3′ |
| SEQ ID NO 66: | 5′-GCAACATAAGACCGCTCAAC-3′ |
| SEQ ID NO 67: | 5′-TAATGGCTGGTAGGTCTGTT-3′ |
| SEQ ID NO 68: | 5′-AACAGACCTACCAGCCATTA-3′ |
| SEQ ID NO 69: | 5′-TAGATAGTCTGGTGGTGGTC-3′ |
| SEQ ID NO 70: | 5′-GACCACCACCAGACTATCTA-3′ |
| SEQ ID NO 71: | 5′-TCGAGTGCTGTTAGGCTACT-3′ |
| SEQ ID NO 72: | 5′-AGTAGCCTAACAGCACTCGA-3′ |
| SEQ ID NO 73: | 5′-TCGTTAGTTCGTTGTTGTCG-3′ |
| SEQ ID NO 74: | 5′-CGACAACAACGAACTAACGA-3′ |
| SEQ ID NO 75: | 5′-TGCGTTGTTATGTCTTGTTG-3′ |
| SEQ ID NO 76: | 5′-CAACAAGACATAACAACGCA-3′ |
| SEQ ID NO 77: | 5′-TGGCTGGTAAGTTGTTATGG-3′ |
| SEQ ID NO 78: | 5′-CCATAACAACTTACCAGCCA-3′ |
| SEQ ID NO 79: | 5′-TGTAGATTGCTTGTCGGTCT-3′ |
| SEQ ID NO 80: | 5′-AGACCGACAAGCAATCTACA-3′ |
| SEQ ID NO 81: | 5′-TGTATTAGCTGATGGCTTGC-3′ |
| SEQ ID NO 82: | 5′-GCAAGCCATCAGCTAATACA-3′ |
| SEQ ID NO 83: | 5′-TGTTAGTGCGGAGTAGGAGT-3′ |
| SEQ ID NO 84: | 5′-ACTCCTACTCCGCACTAACA-3′ |
| SEQ ID NO 85: | 5′-TGTTGTTAGTGCTTGAGGAT-3′ |
| SEQ ID NO 86: | 5′-ATCCTCAAGCACTAACAACA-3′ |
| SEQ ID NO 87: | 5′-TTCGGTCGTTAGCTGTTAGA-3′ |
| SEQ ID NO 88: | 5′-TCTAACAGCTAACGACCGAA-3′ |
| SEQ ID NO 89: | 5′-TTCTGCGGTCGTATGTAGTG-3′ |
| SEQ ID NO 90: | 5′-CACTACATACGACCGCAGAA-3′ |
| SEQ ID NO 91: | 5′-TTGACGGTGGTGAGCTTATT-3′ |
| SEQ ID NO 92: | 5′-AATAAGCTCACCACCGTCAA-3′ |
| SEQ ID NO 93: | 5′-TTGATCTGGATTGTTGACTG-3′ |
| SEQ ID NO 94: | 5′-CAGTCAACAATCCAGATCAA-3′ |
| SEQ ID NO 95: | 5′-TTGCTTCTGAGGATGTTCTG-3′ |
| SEQ ID NO 96: | 5′-CAGAACATCCTCAGAAGCAA-3′ |
| SEQ ID NO 97: | 5′-TTGTAGTAGGTCGTTGCTTG-3′ |
| SEQ ID NO 98: | 5′-CAAGCAACGACCTACTACAA-3′ |
| SEQ ID NO 99: | 5′-ACGTAGTCTGATGGAGTTCA-3′ |
| SEQ ID NO 100: | 5′-TGAACTCCATCAGACTACGT-3′ |
| SEQ ID NO 101: | 5′-AGAAGTGAGTTGCTTCTGCG-3′ |
| SEQ ID NO 102: | 5′-CGCAGAAGCAACTCACTTCT-3′ |
| SEQ ID NO 103: | 5′-AGGCTGCGTTATGACTAGTG-3′ |
| SEQ ID NO 104: | 5′-CACTAGTCATAACGCAGCCT-3′ |
| SEQ ID NO 105: | 5′-ATCTAGGCGTGGCTATTCTG-3′ |
| SEQ ID NO 106: | 5′-CAGAATAGCCACGCCTAGAT-3′ |
| SEQ ID NO 107: | 5′-CAAGTCTGTTGATAGCGGTG-3′ |
| SEQ ID NO 108: | 5′-CACCGCTATCAACAGACTTG-3′ |
| SEQ ID NO 109: | 5′-CTGGTGCGTATCTGTTGTAT-3′ |
| SEQ ID NO 110: | 5′-ATACAACAGATACGCACCAG-3′ |
| SEQ ID NO 111: | 5′-CTGTATGGAGTTGTTCGATC-3′ |
| SEQ ID NO 112: | 5′-GATCGAACAACTCCATACAG-3′ |
| SEQ ID NO 113: | 5′-GATACATTGCTTAGTGCGGT-3′ |
| SEQ ID NO 114: | 5′-ACCGCACTAAGCAATGTATC-3′ |
| SEQ ID NO 115: | 5′-GGATGAGTACGTTGCTTGAC-3′ |
| SEQ ID NO 116: | 5′-GTCAAGCAACGTACTCATCC-3′ |
| SEQ ID NO 117: | 5′-GGTCTTGAGTGGTCTTACGT-3′ |
| SEQ ID NO 118: | 5′-ACGTAAGACCACTCAAGACC-3′ |
| SEQ ID NO 119: | 5′-GGTGCGTAGGTCATTCTAAG-3′ |
| SEQ ID NO 120: | 5′-CTTAGAATGACCTACGCACC-3′ |
| SEQ ID NO 121: | 5′-GTATCTGTTGTCTTGTCGTG-3′ |
| SEQ ID NO 122: | 5′-CACGACAAGACAACAGATAC-3′ |
| SEQ ID NO 123: | 5′-GTGACGTTGACGTGGTACTT-3′ |
| SEQ ID NO 124: | 5′-AAGTACCACGTCAACGTCAC-3′ |
| SEQ ID NO 125: | 5′-GTGCTTGCTGGTCATAGGTA-3′ |
| SEQ ID NO 126: | 5′-TACCTATGACCAGCAAGCAC-3′ |
| SEQ ID NO 127: | 5′-GTGGCTTGTCAGATAGTGAG-3′ |
| SEQ ID NO 128: | 5′-CTCACTATCTGACAAGCCAC-3′ |
| SEQ ID NO 129: | 5′-GTTGACTGATGGATGGATTG-3′ |
| SEQ ID NO 130: | 5′-CAATCCATCCATCAGTCAAC-3′ |
| SEQ ID NO 131: | 5′-TACTCAGTGGTAGTTCGGCT-3′ |
| SEQ ID NO 132: | 5′-AGCCGAACTACCACTGAGTA-3′ |
| SEQ ID NO 133: | 5′-TAGAATGGCGTGAGTTGTAG-3′ |
| SEQ ID NO 134: | 5′-CTACAACTCACGCCATTCTA-3′ |
| SEQ ID NO 135: | 5′-TCTTGTTACAGGTGATCGGT-3′ |
| SEQ ID NO 136: | 5′-ACCGATCACCTGTAACAAGA-3′ |
| SEQ ID NO 137: | 5′-TGATCGTTCGTCGTACTAAG-3′ |
| SEQ ID NO 138: | 5′-CTTAGTACGACGAACGATCA-3′ |
| SEQ ID NO 139: | 5′-TGCGTTGAGTTACTTGATCG-3′ |
| SEQ ID NO 140: | 5′-CGATCAAGTAACTCAACGCA-3′ |
| SEQ ID NO 141: | 5′-TGTATCTAGTTGCGGTCTTG-3′ |
| SEQ ID NO 142: | 5′-CAAGACCGCAACTAGATACA-3′ |
| SEQ ID NO 143: | 5′-TGTCGGCGTAGCTTAGTTAG-3′ |
| SEQ ID NO 144: | 5′-CTAACTAAGCTACGCCGACA-3′ |
| SEQ ID NO 145: | 5′-TTAGTCGGTGAGTGATCTGT-3′ |
| SEQ ID NO 146: | 5′-ACAGATCACTCACCGACTAA-3′ |
| SEQ ID NO 147: | 5′-TTGAGTCATGGAGTTCTTGT-3′ |
| SEQ ID NO 148: | 5′-ACAAGAACTCCATGACTCAA-3′ |
| SEQ ID NO 149: | 5′-TTGATGAGTGATGTTGCTTC-3′ |
| SEQ ID NO 150: | 5′-GAAGCAACATCACTCATCAA-3′ |
| SEQ ID NO 151: | 5′-TTGTTCTAGTGAGGTGATCG-3′ |
| SEQ ID NO 152: | 5′-CGATCACCTCACTAGAACAA-3′ |
| SEQ ID NO 153: | 5′-ATGATTCGCGTTCGTCCTAG-3′ |
| SEQ ID NO 154: | 5′-CTAGGACGAACGCGAATCAT-3′ |
| SEQ ID NO 155: | 5′-ATTTCTCGGTCCGGTTATGA-3′ |
| SEQ ID NO 156: | 5′-TCATAACCGGACCGAGAAAT-3′ |
| SEQ ID NO 157: | 5′-CCTTGCGCTATATCGGGATA-3′ |
| SEQ ID NO 158: | 5′-TATCCCGATATAGCGCAAGG-3′ |
| SEQ ID NO 159: | 5′-CGTTATTAGTCCCGTGGGTT-3′ |
| SEQ ID NO 160: | 5′-AACCCACGGGACTAATAACG-3′ |
| SEQ ID NO 161: | 5′-CTTGTTGGGATGTCGGCTAG-3′ |
| SEQ ID NO 162: | 5′-CTAGCCGACATCCCAACAAG-3′ |
| SEQ ID NO 163: | 5′-GAATGGTCGGTGCATCTTGA-3′ |
| SEQ ID NO 164: | 5′-TCAAGATGCACCGACCATTC-3′ |
| SEQ ID NO 165: | 5′-GATCGTGTTCTGCGGCTTAT-3′ |
| SEQ ID NO 166: | 5′-ATAAGCCGCAGAACACGATC-3′ |
| SEQ ID NO 167: | 5′-GATTGGACGTTCAGGTCTGG-3′ |
| SEQ ID NO 168: | 5′-CCAGACCTGAACGTCCAATC-3′ |
| SEQ ID NO 169: | 5′-GCGTGCTTTACCGTTGTATG-3′ |
| SEQ ID NO 170: | 5′-CATACAACGGTAAAGCACGC-3′ |
| SEQ ID NO 171: | 5′-GTAAGTCGCGGTCGTTAGTA-3′ |
| SEQ ID NO 172: | 5′-TACTAACGACCGCGACTTAC-3′ |
| SEQ ID NO 173: | 5′-GTCTAGGTGTACGGTCTTGC-3′ |
| SEQ ID NO 174: | 5′-GCAAGACCGTACACCTAGAC-3′ |
| SEQ ID NO 175: | 5′-TATTCGATACCGCCGTAGAC-3′ |
| SEQ ID NO 176: | 5′-GTCTACGGCGGTATCGAATA-3′ |
| SEQ ID NO 177: | 5′-TGCTCGATTAACGGATACGG-3′ |
| SEQ ID NO 178: | 5′-CCGTATCCGTTAATCGAGCA-3′ |
| SEQ ID NO 179: | 5′-TGTTTCGTTTGCCGGAGTAA-3′ |
| SEQ ID NO 180: | 5′-TTACTCCGGCAAACGAAACA-3′ |
| SEQ ID NO 181: | 5′-TTTCGGTTGCGTCGGATTAT-3′ |
| SEQ ID NO 182: | 5′-ATAATCCGACGCAACCGAAA-3′ |
| SEQ ID NO 183: | 5′-TTTGTCGTCGTAGCGTACTC-3′ |
| SEQ ID NO 184: | 5′-GAGTACGCTACGACGACAAA-3′ |
| SEQ ID NO 185: | 5′-ATAGGATTGACTGCGGGATC-3′ |
| SEQ ID NO 186: | 5′-GATCCCGCAGTCAATCCTAT-3′ |
| SEQ ID NO 187: | 5′-ATGTATCGTTCGAGCGGATT-3′ |
| SEQ ID NO 188: | 5′-AATCCGCTCGAACGATACAT-3′ |
| SEQ ID NO 189: | 5′-ATTCCCGGCTTCTTGTTAGT-3′ |
| SEQ ID NO 190: | 5′-ACTAACAAGAAGCCGGGAAT-3′ |
| SEQ ID NO 191: | 5′-CATAAGCGCGTTATCCGATG-3′ |
| SEQ ID NO 192: | 5′-CATCGGATAACGCGCTTATG-3′ |
| SEQ ID NO 193: | 5′-CCTCGTGTACCTTGTAGTGC-3′ |
| SEQ ID NO 194: | 5′-GCACTACAAGGTACACGAGG-3′ |
| SEQ ID NO 195: | 5′-CCTTGCCCGGTCCTTATATT-3′ |
| SEQ ID NO 196: | 5′-AATATAAGGACCGGGCAAGG-3′ |
| SEQ ID NO 197: | 5′-CGGATTGGCATTGGTGAGTA-3′ |
| SEQ ID NO 198: | 5′-TACTCACCAATGCCAATCCG-3′ |
| SEQ ID NO 199: | 5′-GGAGTCGTCTAGTAGGGCAT-3′ |
| SEQ ID NO 200: | 5′-ATGCCCTACTAGACGACTCC-3′ |
| SEQ ID NO 201: | 5′-GGCTACGTGTAGATTGTCGG-3′ |
| SEQ ID NO 202: | 5′-CCGACAATCTACACGTAGCC-3′ |
| SEQ ID NO 203: | 5′-GGTTATCGGCGTAGTTGACC-3′ |
| SEQ ID NO 204: | 5′-GGTCAACTACGCCGATAACC-3′ |
| SEQ ID NO 205: | 5′-GTAGGTTGGTCGCGTCTTAC-3′ |
| SEQ ID NO 206: | 5′-GTAAGACGCGACCAACCTAC-3′ |
| SEQ ID NO 207: | 5′-GTAGTCATAGGGCCGGTTTG-3′ |
| SEQ ID NO 208: | 5′-CAAACCGGCCCTATGACTAC-3′ |
| SEQ ID NO 209: | 5′-GTAGTGGTCAGGCTTTAGCA-3′ |
| SEQ ID NO 210: | 5′-TGCTAAAGCCTGACCACTAC-3′ |
| SEQ ID NO 211: | 5′-GTATCGGTCAGTCGTAACGT-3′ |
| SEQ ID NO 212: | 5′-ACGTTACGACTGACCGATAC-3′ |
| SEQ ID NO 213: | 5′-GTCTTGTATTGATCCGCCAC-3′ |
| SEQ ID NO 214: | 5′-GTGGCGGATCAATACAAGAC-3′ |
| SEQ ID NO 215: | 5′-GTGAGTCGCTAGTGTGGTAC-3′ |
| SEQ ID NO 216: | 5′-GTACCACACTAGCGACTCAC-3′ |
| SEQ ID NO 217: | 5′-GTGCTGCGTAATTTGCGTAT-3′ |
| SEQ ID NO 218: | 5′-ATACGCAAATTACGCAGCAC-3′ |
| SEQ ID NO 219: | 5′-GTTTAGGGCGTCCGTTCTAT-3′ |
| SEQ ID NO 220: | 5′-ATAGAACGGACGCCCTAAAC-3′ |
| SEQ ID NO 221: | 5′-GTTTAGTTCGTGCATGGTCC-3′ |
| SEQ ID NO 222: | 5′-GGACCATGCACGAACTAAAC-3′ |
| SEQ ID NO 223: | 5′-TAAAGTCGTGCTGTCAGTGG-3′ |
| SEQ ID NO 224: | 5′-CCACTGACAGCACGACTTTA-3′ |
| SEQ ID NO 225: | 5′-TACACGGTTAGCTCGCTATT-3′ |
| SEQ ID NO 226: | 5′-AATAGCGAGCTAACCGTGTA-3′ |
| SEQ ID NO 227: | 5′-TACTGTCGATTCGTCACGTT-3′ |
| SEQ ID NO 228: | 5′-AACGTGACGAATCGACAGTA-3′ |
| SEQ ID NO 229: | 5′-TATTTCGATCCTCTGGTGCA-3′ |
| SEQ ID NO 230: | 5′-TGCACCAGAGGATCGAAATA-3′ |
| SEQ ID NO 231: | 5′-TCCCTCGGTTCAGTGTTATC-3′ |
| SEQ ID NO 232: | 5′-GATAACACTGAACCGAGGGA-3′ |
| SEQ ID NO 233: | 5′-TCGTGGGCCTTATTACTGTG-3′ |
| SEQ ID NO 234: | 5′-CACAGTAATAAGGCCCACGA-3′ |
| SEQ ID NO 235: | 5′-TCGTTGGTGTGTACTTCGTT-3′ |
| SEQ ID NO 236: | 5′-AACGAAGTACACACCAACGA-3′ |
| SEQ ID NO 237: | 5′-TGCTTTCCGTATTGTCTGGG-3′ |
| SEQ ID NO 238: | 5′-CCCAGACAATACGGAAAGCA-3′ |
| SEQ ID NO 239: | 5′-TGGGTAGAGTTGTGCTAGGT-3′ |
| SEQ ID NO 240: | 5′-ACCTAGCACAACTCTACCCA-3′ |
| SEQ ID NO 241: | 5′-TGTACGTGATTGCTGTAGGG-3′ |
| SEQ ID NO 242: | 5′-CCCTACAGCAATCACGTACA-3′ |
| SEQ ID NO 243: | 5′-TGTCTATTAGCATGTGCGGA-3′ |
| SEQ ID NO 244: | 5′-TCCGCACATGCTAATAGACA-3′ |
| SEQ ID NO 245: | 5′-TGTGTTCGCCTCGTTATAGC-3′ |
| SEQ ID NO 246: | 5′-GCTATAACGAGGCGAACACA-3′ |
| SEQ ID NO 247: | 5′-TTAAGGCGGTCTATCGAGGT-3′ |
| SEQ ID NO 248: | 5′-ACCTCGATAGACCGCCTTAA-3′ |
| SEQ ID NO 249: | 5′-TTAGACGGGTTAGTTGCGAT-3′ |
| SEQ ID NO 250: | 5′-ATCGCAACTAACCCGTCTAA-3′ |
| SEQ ID NO 251: | 5′-TTCATTCGAGTAGCGGTTGG-3′ |
| SEQ ID NO 252: | 5′-CCAACCGCTACTCGAATGAA-3′ |
| SEQ ID NO 253: | 5′-TTCCCGTTAGCCGTTATGTG-3′ |
| SEQ ID NO 254: | 5′-CACATAACGGCTAACGGGAA-3′ |
| SEQ ID NO 255: | 5′-TTCTGATTCAGCGTGGAGTT-3′ |
| SEQ ID NO 256: | 5′-AACTCCACGCTGAATCAGAA-3′ |
| SEQ ID NO 257: | 5′-TTGAGTGCTTCGTATTCCGT-3′ |
| SEQ ID NO 258: | 5′-ACGGAATACGAAGCACTCAA-3′ |
| SEQ ID NO 259: | 5′-TTGATGCTCTCGGGTGTATG-3′ |
| SEQ ID NO 260: | 5′-CATACACCCGAGAGCATCAA-3′ |
| SEQ ID NO 261: | 5′-TTGGGTACTCTCGTAGATGC-3′ |
| SEQ ID NO 262: | 5′-GCATCTACGAGAGTACCCAA-3′ |
| SEQ ID NO 263: | 5′-TTGTTTCAGGGTATTGGGCT-3′ |
| SEQ ID NO 264: | 5′-AGCCCAATACCCTGAAACAA-3′ |
| SEQ ID NO 265: | 5′-TTTACTCGTCCGAGCGTATG-3′ |
| SEQ ID NO 266: | 5′-CATACGCTCGGACGAGTAAA-3′ |

Having thus described the invention, it should be apparent that numerous modifications and adaptations may be resorted to without departing from the scope and fair meaning of the instant invention as set forth hereinabove and as described hereinbelow by the claims.

Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. § 112.

What is claimed is:

1. An assay system for an analyte, the system comprising:
a) a solid support; and
b) six or more pairs of oligonucleotides, each oligonucleotide of the pair is at least partially complementary to the other oligonucleotide of the pair, each pair comprises a first oligonucleotide and a second oligonucleotide, the first oligonucleotide comprising an analyte binding agent attached thereto, the second oligonucleotide being immobilized to the solid support; and
wherein the oligonucleotides have a maximum of three consecutive identical nucleotides; and
the oligonucleotides have less than 10% cross-hybridization when used in assays conducted at from about 37° C. to about 45° C.

2. The assay system of claim 1 wherein the oligonucleotides have less than 3% cross-hybridization when used in assays conducted at from about 37° C. to about 45° C.

3. The assay system of claim 1 wherein the oligonucleotides have less than 1% cross-hybridization when used in assays conducted at from about 37° C. to about 45° C.

4. The assay system of claim 1 wherein the oligonucleotides further comprise:
3 or less consecutive self-complementary nucleotides; and
six or less consecutive purines.

5. The assay system of claim 1 wherein each oligonucleotide further comprises:
12 or less consecutive matching nucleotides with any other oligonucleotide; and
12 or less consecutive matching nucleotides with the complement of any other oligonucleotide.

6. The assay system of claim 1 wherein the oligonucleotides have a Tm of from about 54° C. to about 75° C.

7. The assay system of claim 1 wherein each oligonucleotide contains at least 15 consecutive nucleotides from a different one of SEQ ID NOS: 1-266.

8. The assay system of claim 1 wherein each oligonucleotide is a different one of SEQ ID NOS: 1-266.

9. The assay system of claim 8 further comprising at least 12 pairs of oligonucleotides.

10. The assay system of claim 8 further comprising at least 48 pairs of oligonucleotides.

11. The assay system of claim 8 further comprising at least 96 pairs of oligonucleotides.

12. The assay system of claim 8 further comprising 133 pairs of oligonucleotides.

13. The assay system of claim 1, wherein there are different analyte binding agents linked to the second oligonucleotide of each pair, each different analyte binding agent having a specificity for a different analyte in one or more samples.

14. The assay system of claim 1, wherein one or both oligonucleotides from one or more oligonucleotide pairs further comprises a linker molecule or linker nucleic acid sequence attached thereto.

15. The assay system of claim 14 wherein:
the linker molecule is covalently bonded to one oligonucleotide from one or more oligonucleotide pairs;
the linker molecule is covalently bonded to the solid support; and
the linker molecule attaches the oligonucleotide to the solid support.

16. The assay system of claim 1, wherein each of the different oligonucleotides is attached to the surface of the solid support in a different predefined region.

17. The assay system of claim 1, wherein the solid support is a bead.

18. The assay system of claim 1, wherein the analyte is selected from the group consisting of nucleic acids, polypeptides or proteins, carbohydrates, ligands, lipids, steroids, metabolites, drugs of abuse, viral antigens, bacterial antigens and whole cells.

19. The assay system of claim 18, wherein the analyte is a protein or polypeptide selected from the group consisting of antibodies and binding fragments thereof, hormones, lectins, receptors, steroids, cell-surface antigens, cytokines, growth factors, cDNA, mRNA, disease markers, oncogenic markers, and genomic DNA with single nucleotide polymorphism.

20. The assay system of claim 1 provided in the form of a kit for the detection of one or more specific analytes.

21. A set of at least 48 pairs of oligonucleotides for use in a binding assay, each oligonucleotide in the set comprising:
a) a maximum of three consecutive identical nucleotides;
b) less than about 10% cross-hybridization when used in assays conducted at from about 37° C. to about 45° C.

22. The set of at least 48 pairs of oligonucleotides of claim 21 wherein each oligonucleotide further comprises:
a) three or less consecutive self-complementary nucleotides; and
b) six or less consecutive purines.

23. The set of at least 48 pairs of oligonucleotides of claim 22 wherein each oligonucleotide further comprises:
d) 12 or less consecutive matching nucleotides with any other oligonucleotide;
e) 12 or less consecutive matching nucleotides with the complement of any other oligonucleotide;
f) 60 percent or less homology with any other oligonucleotide;
g) 60 percent or less homology with the complement of any other oligonucleotide; and
h) a Tm of from about 54° C. to about 75° C.

24. The set of at least 48 pairs of oligonucleotides of claim 21 wherein each oligonucleotide contains at least 15 consecutive nucleotides from a different one of SEQ ID NOS: 1-266.

25. The set of at least 48 pairs of oligonucleotides of claim 21 wherein each oligonucleotide is a different one of SEQ ID NOS: 1-266.

26. A method of detecting an analyte in a sample, the method comprising the steps of:
a) selecting six or more complementary oligonucleotide pairs, each pair having a first oligonucleotide and a second oligonucleotide, the first oligonucleotide being immobilized to a solid support, the second oligonucleotide comprising an analyte binding agent attached thereto, each nucleotide having at least 15 consecutive nucleotides from SEQ ID NO:1 to SEQ ID NO:266;
b) providing a sample comprising one or more analyte;
c) admixing one or more analytes with the second oligonucleotides under conditions where the analyte binding agents are able to bind to their respective analytes;
d) attaching a detectable label to the second oligonucleotides;
e) admixing the six or more oligonucleotide pairs under conditions that facilitate the selective hybridization of complementary oligonucleotides to form hybridized oligonucleotides with bound analyte or label; and
f) detecting one or more bound analytes or labels.

27. The method of claim 26 wherein at least one of steps (c), (d), (e), and (f) is conducted at a temperature of from about 20° C. to about 55° C.

28. A method for generating a collection of nucleic acid sequences, the method comprising the steps of:
a) generating a plurality of oligonucleotides, each oligonucleotide having a predetermined Tm;
b) selecting one of the generated oligonucleotides;
c) discarding the selected oligonucleotide if the selected oligonucleotide has more than:
i. about 3 consecutive self-complementary nucleotides;
ii. about 3 consecutive identical nucleotides;
iii. about 6 consecutive purines;
d) comparing the selected oligonucleotide with any other oligonucleotides in the collection and discarding the selected oligonucleotide if the selected oligonucleotide has:
i. more than 12 consecutive matching nucleotides with any oligonucleotide in the collection;
ii. more than 12 consecutive matching nucleotides with the complement of any oligonucleotide in the collection; and
e) adding the selected oligonucleotide to the collection if it is not discarded in steps (c) and (d).

29. The method of claim 28 comprising repeating steps (b), (c), (d), and (e) until the collection comprises at least 24 oligonucleotides.

30. The method of claim 28 comprising repeating steps (b), (c), (d), and (e) until the collection comprises at least 96 oligonucleotides.

31. The method of claim 28 comprising repeating steps (b), (c), (d), and (e) until the collection comprises at least 192 oligonucleotides.

32. The method of claim 28 comprising repeating steps (b), (c), (d), and (e) until the collection comprises at least 266 oligonucleotides.

33. The method of claim 28 comprising the additional step of testing the oligonucleotides in the collection for cross-hybridization, and discarding from the collection any oligonucleotide that cross-hybridizes at more than about 10% with any other oligonucleotides in the collection in assays conducted at from about 37° C. to about 45° C.

34. The method of claim 28 comprising the additional step of testing the oligonucleotides in the collection for cross-hybridization, and discarding from the collection any oligonucleotide that cross-hybridizes at more than about 3% with any other oligonucleotides in the collection in assays conducted at from about 37° C. to about 45° C.

35. The method of claim 28 wherein the step of generating comprises generating a library of nucleotide sequences having substantially the same length.

36. A collection of at least 96 oligonucleotides generated by the method of claim 28.

37. An assay system for an analyte comprising:
a) a solid substrate;
b) at least 12 non-complementary oligonucleotides bound to the solid substrate, each oligonucleotide comprising:
i. a Tm of from about 54° C. to about 75° C.; and
ii. less than about 10% cross-hybridization when used in assays conducted at from about 37° C. to about 45° C.

38. The assay system of clam 37 wherein each oligonucleotide further comprises:
a maximum of three consecutive identical nucleotides; and
six or less consecutive purines.

39. The assay system of claim 37 wherein each oligonucleotide further comprises:
12 or less consecutive matching nucleotides with any other oligonucleotide;
12 or less consecutive matching nucleotides with the complement of any other oligonucleotide; and
three or less consecutive self-complementary nucleotides.

40. The assay system of claim 37 wherein each oligonucleotide is a different one of SEQ ID NOS: 1-266.

41. The assay system of claim 37 further comprising at least 48 non-complementary oligonucleotides bound to the solid substrate.


SEQUENCE LISTING (266 sequences)

SEQ ID NO: 1 - DNA, 20 bp, Artificial Sequence
  Synthetic oligonucleotide
acgtgatggtaatagtgctg

SEQ ID NO: 2 - DNA, 20 bp, Artificial Sequence
  Synthetic oligonucleotide
cagcactattaccatcacgt

SEQ ID NO: 3 - DNA, 20 bp, Artificial Sequence
  Synthetic oligonucleotide
acgtggattcgtagtctgag

SEQ ID NO: 4 - DNA, 20 bp, Artificial Sequence
  Synthetic oligonucleotide
ctcagactacgaatccacgt

SEQ ID NO: 5 - DNA, 20 bp, Artificial Sequence
  Synthetic oligonucleotide
agatagtgaatggatggctg

SEQ ID NO: 6 - DNA, 20 bp, Artificial Sequence
  Synthetic oligonucleotide
cagccatccattcactatct

SEQ ID NO: 7 - DNA, 20 bp, Artificial Sequence
  Synthetic oligonucleotide
aggtcgtaggtgcgttagat

SEQ ID NO: 8 - DNA, 20 bp, Artificial Sequence
  Synthetic oligonucleotide
atctaacgcacctacgacct

SEQ ID NO: 9 - DNA, 20 bp, Artificial Sequence
  Synthetic oligonucleotide
agtctgcggattacttgttg

SEQ ID NO: 10 - DNA, 20 bp, Artificial Sequence
  Synthetic Oligonucleotide
caacaagtaatccgcagact

SEQ ID NO: 11 - DNA, 20 bp, Artificial Sequence
  Synthetic Oligonucleotide
atggagtgctgttgattctt

SEQ ID NO: 12 - DNA, 20 bp, Artificial Sequence
  Synthetic Oligonucleotide
aagaatcaacagcactccat

SEQ ID NO: 13 - DNA, 20 bp, Artificial Sequence
  Synthetic Oligonucleotide
atggtacttgcgtgagttgt

SEQ ID NO: 14 - DNA, 20 bp, Artificial Sequence
  Synthetic Oligonucleotide
acaactcacgcaagtaccat

SEQ ID NO: 15 - DNA, 20 bp, Artificial Sequence
  Synthetic Oligonucleotide
atgtctagtgcgtgcttgta

SEQ ID NO: 16 - DNA, 20 bp, Artificial Sequence
  Synthetic oligonucleotide
tacaagcacgcactagacat

SEQ ID NO: 17 - DNA, 20 bp, Artificial Sequence
  Synthetic oligonucleotide
cagtgcttgcgtagaagtct

SEQ ID NO: 18 - DNA, 20 bp, Artificial Sequence
  Synthetic oligonucleotide
agacttctacgcaagcactg

SEQ ID NO: 19 - DNA, 20 bp, Artificial Sequence
  Synthetic oligonucleotide
cggtaggatggattatggat

SEQ ID NO: 20 - DNA, 20 bp, Artificial Sequence
  Synthetic oligonucleotide
atccataatccatcctaccg

SEQ ID NO: 21 - DNA, 20 bp, Artificial Sequence
  Synthetic oligonucleotide
cgtctaggtcttactggcgg

SEQ ID NO: 22 - DNA, 20 bp, Artificial Sequence
  Synthetic oligonucleotide
ccgccagtaagacctagacg

SEQ ID NO: 23 - DNA, 20 bp, Artificial Sequence
  Synthetic oligonucleotide
cgtctgttgtctattctgga

SEQ ID NO: 24 - DNA, 20 bp, Artificial Sequence
  Synthetic oligonucleotide
tccagaatagacaacagacg

SEQ ID NO: 25 - DNA, 20 bp, Artificial Sequence
  Synthetic oligonucleotide
ctgctcggtatgttgctgtt

SEQ ID NO: 26 - DNA, 20 bp, Artificial Sequence
  Synthetic oligonucleotide
aacagcaacataccgagcag

SEQ ID NO: 27 - DNA, 20 bp, Artificial Sequence
  Synthetic oligonucleotide
ctggtctaggtggtcttgct

SEQ ID NO: 28 - DNA, 20 bp, Artificial Sequence
  Synthetic oligonucleotide
agcaagaccacctagaccag

SEQ ID NO: 29 - DNA, 20 bp, Artificial Sequence
  Synthetic oligonucleotide
ctgttgttgcgtgctgttat

SEQ ID NO: 30 - DNA, 20 bp, Artificial Sequence
  Synthetic oligonucleotide
ataacagcacgcaacaacag

SEQ ID NO: 31 - DNA, 20 bp, Artificial Sequence
  Synthetic oligonucleotide
cttacatggtgaggatctgg

SEQ ID NO: 32 - DNA, 20 bp, Artificial Sequence
  Synthetic oligonucleotide
ccagatcctcaccatgtaag

SEQ ID NO: 33 - DNA, 20 bp, Artificial Sequence
  Synthetic oligonucleotide
cttgctggctcgtattgttg

SEQ ID NO: 34 - DNA, 20 bp, Artificial Sequence
  Synthetic oligonucleotide
caacaatacgagccagcaag

SEQ ID NO: 35 - DNA, 20 bp, Artificial Sequence
  Synthetic oligonucleotide
gagcttgttgcttcttgcat

SEQ ID NO: 36 - DNA, 20 bp, Artificial Sequence
  Synthetic oligonucleotide
atgcaagaagcaacaagctc

SEQ ID NO: 37 - DNA, 20 bp, Artificial Sequence
  Synthetic oligonucleotide
gaggagtgcgtctgtatgtt

SEQ ID NO: 38 - DNA, 20 bp, Artificial Sequence
  Synthetic oligonucleotide
aacatacagacgcactcctc

SEQ ID NO: 39 - DNA, 20 bp, Artificial Sequence
  Synthetic oligonucleotide
gaggtggaggtcttgatagg

SEQ ID NO: 40 - DNA, 20 bp, Artificial Sequence
  Synthetic oligonucleotide
cctatcaagacctccacctc

SEQ ID NO: 41 - DNA, 20 bp, Artificial Sequence
  Synthetic oligonucleotide
gctgttcgtatgttgattgt

SEQ ID NO: 42 - DNA, 20 bp, Artificial Sequence
  Synthetic oligonucleotide
acaatcaacatacgaacagc

SEQ ID NO: 43 - DNA, 20 bp, Artificial Sequence
  Synthetic oligonucleotide
ggattgttcggtcttgaagt

SEQ ID NO: 44 - DNA, 20 bp, Artificial Sequence
  Synthetic oligonucleotide
acttcaagaccgaacaatcc

SEQ ID NO: 45 - DNA, 20 bp, Artificial Sequence
  Synthetic oligonucleotide
ggctgtagcgtggtaggtat

SEQ ID NO: 46 - DNA, 20 bp, Artificial Sequence
  Synthetic oligonucleotide
atacctaccacgctacagcc

SEQ ID NO: 47 - DNA, 20 bp, Artificial Sequence
  Synthetic oligonucleotide
ggtagttgttcgttgtctgc

SEQ ID NO: 48 - DNA, 20 bp, Artificial Sequence
  Synthetic oligonucleotide
gcagacaacgaacaactacc

SEQ ID NO: 49 - DNA, 20 bp, Artificial Sequence
  Synthetic oligonucleotide
ggtggtgatgtagatggtaa

SEQ ID NO: 50 - DNA, 20 bp, Artificial Sequence
  Synthetic oligonucleotide
ttaccatctacatcaccacc

SEQ ID NO: 51 - DNA, 20 bp, Artificial Sequence
  Synthetic oligonucleotide
gtatgagttgcgtggatgtt

SEQ ID NO: 52 - DNA, 20 bp, Artificial Sequence
  Synthetic oligonucleotide
aacatccacgcaactcatac

SEQ ID NO: 53 - DNA, 20 bp, Artificial Sequence
  Synthetic oligonucleotide
gtattcgtggagttgagtgg

SEQ ID NO: 54 - DNA, 20 bp, Artificial Sequence
  Synthetic oligonucleotide
ccactcaactccacgaatac

SEQ ID NO: 55 - DNA, 20 bp, Artificial Sequence
  Synthetic oligonucleotide
gtcttgtcgtcggtagttgt

SEQ ID NO: 56 - DNA, 20 bp, Artificial Sequence
  Synthetic oligonucleotide
acaactaccgacgacaagac

SEQ ID NO: 57 - DNA, 20 bp, Artificial Sequence
  Synthetic oligonucleotide
gtgagtgcggtatgttgtct

SEQ ID NO: 58 - DNA, 20 bp, Artificial Sequence
  Synthetic oligonucleotide
agacaacataccgcactcac

SEQ ID NO: 59 - DNA, 20 bp, Artificial Sequence
  Synthetic oligonucleotide
gtgatgtctgttgatggatc

SEQ ID NO: 60 - DNA, 20 bp, Artificial Sequence
  Synthetic oligonucleotide
gatccatcaacagacatcac

SEQ ID NO: 61 - DNA, 20 bp, Artificial Sequence
  Synthetic oligonucleotide
gtgctggtcttgttatgttg

SEQ ID NO: 62 - DNA, 20 bp, Artificial Sequence
  Synthetic oligonucleotide
caacataacaagaccagcac

SEQ ID NO: 63 - DNA, 20 bp, Artificial Sequence
  Synthetic oligonucleotide
gttactgcgtatggattgcg

SEQ ID NO: 64 - DNA, 20 bp, Artificial Sequence
  Synthetic oligonucleotide
cgcaatccatacgcagtaac

SEQ ID NO: 65 - DNA, 20 bp, Artificial Sequence
  Synthetic oligonucleotide
gttgagcggtcttatgttgc

SEQ ID NO: 66 - DNA, 20 bp, Artificial Sequence
  Synthetic oligonucleotide
gcaacataagaccgctcaac

SEQ ID NO: 67 - DNA, 20 bp, Artificial Sequence
  Synthetic oligonucleotide
taatggctggtaggtctgtt

SEQ ID NO: 68 - DNA, 20 bp, Artificial Sequence
  Synthetic oligonucleotide
aacagacctaccagccatta

SEQ ID NO: 69 - DNA, 20 bp, Artificial Sequence
  Synthetic oligonucleotide
tagatagtctggtggtggtc

SEQ ID NO: 70 - DNA, 20 bp, Artificial Sequence
  Synthetic oligonucleotide
gaccaccaccagactatcta

SEQ ID NO: 71 - DNA, 20 bp, Artificial Sequence
  Synthetic oligonucleotide
tcgagtgctgttaggctact

SEQ ID NO: 72 - DNA, 20 bp, Artificial Sequence
  Synthetic oligonucleotide
agtagcctaacagcactcga

SEQ ID NO: 73 - DNA, 20 bp, Artificial Sequence
  Synthetic oligonucleotide
tcgttagttcgttgttgtcg

SEQ ID NO: 74 - DNA, 20 bp, Artificial Sequence
  Synthetic oligonucleotide
cgacaacaacgaactaacga

SEQ ID NO: 75 - DNA, 20 bp, Artificial Sequence
  Synthetic oligonucleotide
tgcgttgttatgtcttgttg

SEQ ID NO: 76 - DNA, 20 bp, Artificial Sequence
  Synthetic oligonucleotide
caacaagacataacaacgca

SEQ ID NO: 77 - DNA, 20 bp, Artificial Sequence
  Synthetic oligonucleotide
tggctggtaagttgttatgg

SEQ ID NO: 78 - DNA, 20 bp, Artificial Sequence
  Synthetic oligonucleotide
ccataacaacttaccagcca

SEQ ID NO: 79 - DNA, 20 bp, Artificial Sequence
  Synthetic oligonucleotide
tgtagattgcttgtcggtct

SEQ ID NO: 80 - DNA, 20 bp, Artificial Sequence
  Synthetic oligonucleotide
agaccgacaagcaatctaca

SEQ ID NO: 81 - DNA, 20 bp, Artificial Sequence
  Synthetic oligonucleotide
tgtattagctgatggcttgc

SEQ ID NO: 82 - DNA, 20 bp, Artificial Sequence
  Synthetic oligonucleotide
gcaagccatcagctaataca

SEQ ID NO: 83 - DNA, 20 bp, Artificial Sequence
  Synthetic oligonucleotide
tgttagtgcggagtaggagt

SEQ ID NO: 84 - DNA, 20 bp, Artificial Sequence
  Synthetic oligonucleotide
actcctactccgcactaaca

SEQ ID NO: 85 - DNA, 20 bp, Artificial Sequence
  Synthetic oligonucleotide
tgttgttagtgcttgaggat

SEQ ID NO: 86 - DNA, 20 bp, Artificial Sequence
  Synthetic oligonucleotide
atcctcaagcactaacaaca

SEQ ID NO: 87 - DNA, 20 bp, Artificial Sequence
  Synthetic oligonucleotide
ttcggtcgttagctgttaga

SEQ ID NO: 88 - DNA, 20 bp, Artificial Sequence
  Synthetic oligonucleotide
tctaacagctaacgaccgaa

SEQ ID NO: 89 - DNA, 20 bp, Artificial Sequence
  Synthetic oligonucleotide
ttctgcggtcgtatgtagtg

SEQ ID NO: 90 - DNA, 20 bp, Artificial Sequence
  Synthetic oligonucleotide
cactacatacgaccgcagaa

SEQ ID NO: 91 - DNA, 20 bp, Artificial Sequence
  Synthetic oligonucleotide
ttgacggtggtgagcttatt

SEQ ID NO: 92 - DNA, 20 bp, Artificial Sequence
  Synthetic oligonucleotide
aataagctcaccaccgtcaa

SEQ ID NO: 93 - DNA, 20 bp, Artificial Sequence
  Synthetic oligonucleotide
ttgatctggattgttgactg

SEQ ID NO: 94 - DNA, 20 bp, Artificial Sequence
  Synthetic oligonucleotide
cagtcaacaatccagatcaa

SEQ ID NO: 95 - DNA, 20 bp, Artificial Sequence
  Synthetic oligonucleotide
ttgcttctgaggatgttctg

SEQ ID NO: 96 - DNA, 20 bp, Artificial Sequence
  Synthetic oligonucleotide
cagaacatcctcagaagcaa

SEQ ID NO: 97 - DNA, 20 bp, Artificial Sequence
  Synthetic oligonucleotide
ttgtagtaggtcgttgcttg

SEQ ID NO: 98 - DNA, 20 bp, Artificial Sequence
  Synthetic oligonucleotide
caagcaacgacctactacaa

SEQ ID NO: 99 - DNA, 20 bp, Artificial Sequence
  Synthetic oligonucleotide
acgtagtctgatggagttca

SEQ ID NO: 100 - DNA, 20 bp, Artificial Sequence
  Synthetic oligonucleotide
tgaactccatcagactacgt

SEQ ID NO: 101 - DNA, 20 bp, Artificial Sequence
  Synthetic oligonucleotide
agaagtgagttgcttctgcg

SEQ ID NO: 102 - DNA, 20 bp, Artificial Sequence
  Synthetic oligonucleotide
cgcagaagcaactcacttct

SEQ ID NO: 103 - DNA, 20 bp, Artificial Sequence
  Synthetic oligonucleotide
aggctgcgttatgactagtg

SEQ ID NO: 104 - DNA, 20 bp, Artificial Sequence
  Synthetic oligonucleotide
cactagtcataacgcagcct

SEQ ID NO: 105 - DNA, 20 bp, Artificial Sequence
  Synthetic oligonucleotide
atctaggcgtggctattctg

SEQ ID NO: 106 - DNA, 20 bp, Artificial Sequence
  Synthetic oligonucleotide
cagaatagccacgcctagat

SEQ ID NO: 107 - DNA, 20 bp, Artificial Sequence
  Synthetic oligonucleotide
caagtctgttgatagcggtg

SEQ ID NO: 108 - DNA, 20 bp, Artificial Sequence
  Synthetic oligonucleotide
caccgctatcaacagacttg

SEQ ID NO: 109 - DNA, 20 bp, Artificial Sequence
  Synthetic oligonucleotide
ctggtgcgtatctgttgtat

SEQ ID NO: 110 - DNA, 20 bp, Artificial Sequence
  Synthetic oligonucleotide
atacaacagatacgcaccag

SEQ ID NO: 111 - DNA, 20 bp, Artificial Sequence
  Synthetic oligonucleotide
ctgtatggagttgttcgatc

SEQ ID NO: 112 - DNA, 20 bp, Artificial Sequence
  Synthetic oligonucleotide
gatcgaacaactccatacag

SEQ ID NO: 113 - DNA, 20 bp, Artificial Sequence
  Synthetic oligonucleotide
gatacattgcttagtgcggt

SEQ ID NO: 114 - DNA, 20 bp, Artificial Sequence
  Synthetic oligonucleotide
accgcactaagcaatgtatc

SEQ ID NO: 115 - DNA, 20 bp, Artificial Sequence
  Synthetic oligonucleotide
ggatgagtacgttgcttgac

SEQ ID NO: 116 - DNA, 20 bp, Artificial Sequence
  Synthetic oligonucleotide
gtcaagcaacgtactcatcc

SEQ ID NO: 117 - DNA, 20 bp, Artificial Sequence
  Synthetic oligonucleotide
ggtcttgagtggtcttacgt

SEQ ID NO: 118 - DNA, 20 bp, Artificial Sequence
  Synthetic oligonucleotide
acgtaagaccactcaagacc

SEQ ID NO: 119 - DNA, 20 bp, Artificial Sequence
  Synthetic oligonucleotide
ggtgcgtaggtcattctaag

SEQ ID NO: 120 - DNA, 20 bp, Artificial Sequence
  Synthetic oligonucleotide
cttagaatgacctacgcacc

SEQ ID NO: 121 - DNA, 20 bp, Artificial Sequence
  Synthetic oligonucleotide
gtatctgttgtcttgtcgtg

SEQ ID NO: 122 - DNA, 20 bp, Artificial Sequence
  Synthetic oligonucleotide
cacgacaagacaacagatac

SEQ ID NO: 123 - DNA, 20 bp, Artificial Sequence
  Synthetic oligonucleotide
gtgacgttgacgtggtactt

SEQ ID NO: 124 - DNA, 20 bp, Artificial Sequence
  Synthetic oligonucleotide
aagtaccacgtcaacgtcac

SEQ ID NO: 125 - DNA, 20 bp, Artificial Sequence
  Synthetic oligonucleotide
gtgcttgctggtcataggta

SEQ ID NO: 126 - DNA, 20 bp, Artificial Sequence
  Synthetic oligonucleotide
tacctatgaccagcaagcac

SEQ ID NO: 127 - DNA, 20 bp, Artificial Sequence
  Synthetic oligonucleotide
gtggcttgtcagatagtgag

SEQ ID NO: 128 - DNA, 20 bp, Artificial Sequence
  Synthetic oligonucleotide
ctcactatctgacaagccac

SEQ ID NO: 129 - DNA, 20 bp, Artificial Sequence
  Synthetic oligonucleotide
gttgactgatggatggattg

SEQ ID NO: 130 - DNA, 20 bp, Artificial Sequence
  Synthetic oligonucleotide
caatccatccatcagtcaac

SEQ ID NO: 131 - DNA, 20 bp, Artificial Sequence
  Synthetic oligonucleotide
tactcagtggtagttcggct

SEQ ID NO: 132 - DNA, 20 bp, Artificial Sequence
  Synthetic oligonucleotide
agccgaactaccactgagta

SEQ ID NO: 133 - DNA, 20 bp, Artificial Sequence
  Synthetic oligonucleotide
tagaatggcgtgagttgtag

SEQ ID NO: 134 - DNA, 20 bp, Artificial Sequence
  Synthetic oligonucleotide
ctacaactcacgccattcta

SEQ ID NO: 135 - DNA, 20 bp, Artificial Sequence
  Synthetic oligonucleotide
tcttgttacaggtgatcggt

SEQ ID NO: 136 - DNA, 20 bp, Artificial Sequence
  Synthetic oligonucleotide
accgatcacctgtaacaaga

SEQ ID NO: 137 - DNA, 20 bp, Artificial Sequence
  Synthetic oligonucleotide
tgatcgttcgtcgtactaag

SEQ ID NO: 138 - DNA, 20 bp, Artificial Sequence
  Synthetic oligonucleotide
cttagtacgacgaacgatca

SEQ ID NO: 139 - DNA, 20 bp, Artificial Sequence
  Synthetic oligonucleotide
tgcgttgagttacttgatcg

SEQ ID NO: 140 - DNA, 20 bp, Artificial Sequence
  Synthetic oligonucleotide
cgatcaagtaactcaacgca

SEQ ID NO: 141 - DNA, 20 bp, Artificial Sequence
  Synthetic oligonucleotide
tgtatctagttgcggtcttg

SEQ ID NO: 142 - DNA, 20 bp, Artificial Sequence
  Synthetic oligonucleotide
caagaccgcaactagataca

SEQ ID NO: 143 - DNA, 20 bp, Artificial Sequence
  Synthetic oligonucleotide
tgtcggcgtagcttagttag

SEQ ID NO: 144 - DNA, 20 bp, Artificial Sequence
  Synthetic oligonucleotide
ctaactaagctacgccgaca

SEQ ID NO: 145 - DNA, 20 bp, Artificial Sequence
  Synthetic oligonucleotide
ttagtcggtgagtgatctgt

SEQ ID NO: 146 - DNA, 20 bp, Artificial Sequence
  Synthetic oligonucleotide
acagatcactcaccgactaa

SEQ ID NO: 147 - DNA, 20 bp, Artificial Sequence
  Synthetic oligonucleotide
ttgagtcatggagttcttgt

SEQ ID NO: 148 - DNA, 20 bp, Artificial Sequence
  Synthetic oligonucleotide
acaagaactccatgactcaa

SEQ ID NO: 149 - DNA, 20 bp, Artificial Sequence
  Synthetic oligonucleotide
ttgatgagtgatgttgcttc

SEQ ID NO: 150 - DNA, 20 bp, Artificial Sequence
  Synthetic oligonucleotide
gaagcaacatcactcatcaa

SEQ ID NO: 151 - DNA, 20 bp, Artificial Sequence
  Synthetic oligonucleotide
ttgttctagtgaggtgatcg

SEQ ID NO: 152 - DNA, 20 bp, Artificial Sequence
  Synthetic oligonucleotide
cgatcacctcactagaacaa

SEQ ID NO: 153 - DNA, 20 bp, Artificial Sequence
  Synthetic oligonucleotide
atgattcgcgttcgtcctag

SEQ ID NO: 154 - DNA, 20 bp, Artificial Sequence
  Synthetic oligonucleotide
ctaggacgaacgcgaatcat

SEQ ID NO: 155 - DNA, 20 bp, Artificial Sequence
  Synthetic oligonucleotide
atttctcggtccggttatga

SEQ ID NO: 156 - DNA, 20 bp, Artificial Sequence
  Synthetic oligonucleotide
tcataaccggaccgagaaat

SEQ ID NO: 157 - DNA, 20 bp, Artificial Sequence
  Synthetic oligonucleotide
ccttgcgctatatcgggata

SEQ ID NO: 158 - DNA, 20 bp, Artificial Sequence
  Synthetic oligonucleotide
tatcccgatatagcgcaagg

SEQ ID NO: 159 - DNA, 20 bp, Artificial Sequence
  Synthetic oligonucleotide
cgttattagtcccgtgggtt

SEQ ID NO: 160 - DNA, 20 bp, Artificial Sequence
  Synthetic oligonucleotide
aacccacgggactaataacg

SEQ ID NO: 161 - DNA, 20 bp, Artificial Sequence
  Synthetic oligonucleotide
cttgttgggatgtcggctag

SEQ ID NO: 162 - DNA, 20 bp, Artificial Sequence
  Synthetic oligonucleotide
ctagccgacatcccaacaag

SEQ ID NO: 163 - DNA, 20 bp, Artificial Sequence
  Synthetic oligonucleotide
gaatggtcggtgcatcttga

SEQ ID NO: 164 - DNA, 20 bp, Artificial Sequence
  Synthetic oligonucleotide
tcaagatgcaccgaccattc

SEQ ID NO: 165 - DNA, 20 bp, Artificial Sequence
  Synthetic oligonucleotide
gatcgtgttctgcggcttat

SEQ ID NO: 166 - DNA, 20 bp, Artificial Sequence
  Synthetic oligonucleotide
ataagccgcagaacacgatc

SEQ ID NO: 167 - DNA, 20 bp, Artificial Sequence
  Synthetic oligonucleotide
gattggacgttcaggtctgg

SEQ ID NO: 168 - DNA, 20 bp, Artificial Sequence
  Synthetic oligonucleotide
ccagacctgaacgtccaatc

SEQ ID NO: 169 - DNA, 20 bp, Artificial Sequence
  Synthetic oligonucleotide
gcgtgctttaccgttgtatg

SEQ ID NO: 170 - DNA, 20 bp, Artificial Sequence
  Synthetic oligonucleotide
catacaacggtaaagcacgc

SEQ ID NO: 171 - DNA, 20 bp, Artificial Sequence
  Synthetic oligonucleotide
gtaagtcgcggtcgttagta

SEQ ID NO: 172 - DNA, 20 bp, Artificial Sequence
  Synthetic oligonucleotide
tactaacgaccgcgacttac

SEQ ID NO: 173 - DNA, 20 bp, Artificial Sequence
  Synthetic oligonucleotide
gtctaggtgtacggtcttgc

SEQ ID NO: 174 - DNA, 20 bp, Artificial Sequence
  Synthetic oligonucleotide
gcaagaccgtacacctagac

SEQ ID NO: 175 - DNA, 20 bp, Artificial Sequence
  Synthetic oligonucleotide
tattcgataccgccgtagac

SEQ ID NO: 176 - DNA, 20 bp, Artificial Sequence
  Synthetic oligonucleotide
gtctacggcggtatcgaata

SEQ ID NO: 177 - DNA, 20 bp, Artificial Sequence
  Synthetic oligonucleotide
tgctcgattaacggatacgg

SEQ ID NO: 178 - DNA, 20 bp, Artificial Sequence
  Synthetic oligonucleotide
ccgtatccgttaatcgagca

SEQ ID NO: 179 - DNA, 20 bp, Artificial Sequence
  Synthetic oligonucleotide
tgtttcgtttgccggagtaa

SEQ ID NO: 180 - DNA, 20 bp, Artificial Sequence
  Synthetic oligonucleotide
ttactccggcaaacgaaaca

SEQ ID NO: 181 - DNA, 20 bp, Artificial Sequence
  Synthetic oligonucleotide
tttcggttgcgtcggattat

SEQ ID NO: 182 - DNA, 20 bp, Artificial Sequence
  Synthetic oligonucleotide
ataatccgacgcaaccgaaa

SEQ ID NO: 183 - DNA, 20 bp, Artificial Sequence
  Synthetic oligonucleotide
tttgtcgtcgtagcgtactc

SEQ ID NO: 184 - DNA, 20 bp, Artificial Sequence
  Synthetic oligonucleotide
gagtacgctacgacgacaaa

SEQ ID NO: 185 - DNA, 20 bp, Artificial Sequence
  Synthetic oligonucleotide
ataggattgactgcgggatc

SEQ ID NO: 186 - DNA, 20 bp, Artificial Sequence
  Synthetic oligonucleotide
gatcccgcagtcaatcctat

SEQ ID NO: 187 - DNA, 20 bp, Artificial Sequence
  Synthetic oligonucleotide
atgtatcgttcgagcggatt

SEQ ID NO: 188 - DNA, 20 bp, Artificial Sequence
  Synthetic oligonucleotide
aatccgctcgaacgatacat

SEQ ID NO: 189 - DNA, 20 bp, Artificial Sequence
  Synthetic oligonucleotide
attcccggcttcttgttagt

SEQ ID NO: 190 - DNA, 20 bp, Artificial Sequence
  Synthetic oligonucleotide
actaacaagaagccgggaat

SEQ ID NO: 191 - DNA, 20 bp, Artificial Sequence
  Synthetic oligonucleotide
cataagcgcgttatccgatg

SEQ ID NO: 192 - DNA, 20 bp, Artificial Sequence
  Synthetic oligonucleotide
catcggataacgcgcttatg

SEQ ID NO: 193 - DNA, 20 bp, Artificial Sequence
  Synthetic oligonucleotide
cctcgtgtaccttgtagtgc

SEQ ID NO: 194 - DNA, 20 bp, Artificial Sequence
  Synthetic oligonucleotide
gcactacaaggtacacgagg

SEQ ID NO: 195 - DNA, 20 bp, Artificial Sequence
  Synthetic oligonucleotide
ccttgcccggtccttatatt

SEQ ID NO: 196 - DNA, 20 bp, Artificial Sequence
  Synthetic oligonucleotide
aatataaggaccgggcaagg

SEQ ID NO: 197 - DNA, 20 bp, Artificial Sequence
  Synthetic oligonucleotide
cggattggcattggtgagta

SEQ ID NO: 198 - DNA, 20 bp, Artificial Sequence
  Synthetic oligonucleotide
tactcaccaatgccaatccg

SEQ ID NO: 199 - DNA, 20 bp, Artificial Sequence
  Synthetic oligonucleotide
ggagtcgtctagtagggcat

SEQ ID NO: 200 - DNA, 20 bp, Artificial Sequence
  Synthetic oligonucleotide
atgccctactagacgactcc

SEQ ID NO: 201 - DNA, 20 bp, Artificial Sequence
  Synthetic oligonucleotide
ggctacgtgtagattgtcgg

SEQ ID NO: 202 - DNA, 20 bp, Artificial Sequence
  Synthetic oligonucleotide
ccgacaatctacacgtagcc

SEQ ID NO: 203 - DNA, 20 bp, Artificial Sequence
  Synthetic oligonucleotide
ggttatcggcgtagttgacc

SEQ ID NO: 204 - DNA, 20 bp, Artificial Sequence
  Synthetic oligonucleotide
ggtcaactacgccgataacc

SEQ ID NO: 205 - DNA, 20 bp, Artificial Sequence
  Synthetic oligonucleotide
gtaggttggtcgcgtcttac

SEQ ID NO: 206 - DNA, 20 bp, Artificial Sequence
  Synthetic oligonucleotide
gtaagacgcgaccaacctac

SEQ ID NO: 207 - DNA, 20 bp, Artificial Sequence
  Synthetic oligonucleotide
gtagtcatagggccggtttg

SEQ ID NO: 208 - DNA, 20 bp, Artificial Sequence
  Synthetic oligonucleotide
caaaccggccctatgactac

SEQ ID NO: 209 - DNA, 20 bp, Artificial Sequence
  Synthetic oligonucleotide
gtagtggtcaggctttagca

SEQ ID NO: 210 - DNA, 20 bp, Artificial Sequence
  Synthetic oligonucleotide
tgctaaagcctgaccactac

SEQ ID NO: 211 - DNA, 20 bp, Artificial Sequence
  Synthetic oligonucleotide
gtatcggtcagtcgtaacgt

SEQ ID NO: 212 - DNA, 20 bp, Artificial Sequence
  Synthetic oligonucleotide
acgttacgactgaccgatac

SEQ ID NO: 213 - DNA, 20 bp, Artificial Sequence
  Synthetic oligonucleotide
gtcttgtattgatccgccac

SEQ ID NO: 214 - DNA, 20 bp, Artificial Sequence
  Synthetic oligonucleotide
gtggcggatcaatacaagac

SEQ ID NO: 215 - DNA, 20 bp, Artificial Sequence
  Synthetic oligonucleotide
gtgagtcgctagtgtggtac

SEQ ID NO: 216 - DNA, 20 bp, Artificial Sequence
  Synthetic oligonucleotide
gtaccacactagcgactcac

SEQ ID NO: 217 - DNA, 20 bp, Artificial Sequence
  Synthetic oligonucleotide
gtgctgcgtaatttgcgtat

SEQ ID NO: 218 - DNA, 20 bp, Artificial Sequence
  Synthetic oligonucleotide
atacgcaaattacgcagcac

SEQ ID NO: 219 - DNA, 20 bp, Artificial Sequence
  Synthetic oligonucleotide
gtttagggcgtccgttctat

SEQ ID NO: 220 - DNA, 20 bp, Artificial Sequence
  Synthetic oligonucleotide
atagaacggacgccctaaac

SEQ ID NO: 221 - DNA, 20 bp, Artificial Sequence
  Synthetic oligonucleotide
gtttagttcgtgcatggtcc

SEQ ID NO: 222 - DNA, 20 bp, Artificial Sequence
  Synthetic oligonucleotide
ggaccatgcacgaactaaac

SEQ ID NO: 223 - DNA, 20 bp, Artificial Sequence
  Synthetic oligonucleotide
taaagtcgtgctgtcagtgg

SEQ ID NO: 224 - DNA, 20 bp, Artificial Sequence
  Synthetic oligonucleotide
ccactgacagcacgacttta

SEQ ID NO: 225 - DNA, 20 bp, Artificial Sequence
  Synthetic oligonucleotide
tacacggttagctcgctatt

SEQ ID NO: 226 - DNA, 20 bp, Artificial Sequence
  Synthetic oligonucleotide
aatagcgagctaaccgtgta

SEQ ID NO: 227 - DNA, 20 bp, Artificial Sequence
  Synthetic oligonucleotide
tactgtcgattcgtcacgtt

SEQ ID NO: 228 - DNA, 20 bp, Artificial Sequence
  Synthetic oligonucleotide
aacgtgacgaatcgacagta

SEQ ID NO: 229 - DNA, 20 bp, Artificial Sequence
  Synthetic oligonucleotide
tatttcgatcctctggtgca

SEQ ID NO: 230 - DNA, 20 bp, Artificial Sequence
  Synthetic oligonucleotide
tgcaccagaggatcgaaata

SEQ ID NO: 231 - DNA, 20 bp, Artificial Sequence
  Synthetic oligonucleotide
tccctcggttcagtgttatc

SEQ ID NO: 232 - DNA, 20 bp, Artificial Sequence
  Synthetic oligonucleotide
gataacactgaaccgaggga

SEQ ID NO: 233 - DNA, 20 bp, Artificial Sequence
  Synthetic oligonucleotide
tcgtgggccttattactgtg

SEQ ID NO: 234 - DNA, 20 bp, Artificial Sequence
  Synthetic oligonucleotide
cacagtaataaggcccacga

SEQ ID NO: 235 - DNA, 20 bp, Artificial Sequence
  Synthetic oligonucleotide
tcgttggtgtgtacttcgtt

SEQ ID NO: 236 - DNA, 20 bp, Artificial Sequence
  Synthetic oligonucleotide
aacgaagtacacaccaacga

SEQ ID NO: 237 - DNA, 20 bp, Artificial Sequence
  Synthetic oligonucleotide
tgctttccgtattgtctggg

SEQ ID NO: 238 - DNA, 20 bp, Artificial Sequence
  Synthetic oligonucleotide
cccagacaatacggaaagca

SEQ ID NO: 239 - DNA, 20 bp, Artificial Sequence
  Synthetic oligonucleotide
tgggtagagttgtgctaggt

SEQ ID NO: 240 - DNA, 20 bp, Artificial Sequence
  Synthetic oligonucleotide
acctagcacaactctaccca

SEQ ID NO: 241 - DNA, 20 bp, Artificial Sequence
  Synthetic oligonucleotide
tgtacgtgattgctgtaggg

SEQ ID NO: 242 - DNA, 20 bp, Artificial Sequence
  Synthetic oligonucleotide
ccctacagcaatcacgtaca

SEQ ID NO: 243 - DNA, 20 bp, Artificial Sequence
  Synthetic oligonucleotide
tgtctattagcatgtgcgga

SEQ ID NO: 244 - DNA, 20 bp, Artificial Sequence
  Synthetic oligonucleotide
tccgcacatgctaatagaca

SEQ ID NO: 245 - DNA, 20 bp, Artificial Sequence
  Synthetic oligonucleotide
tgtgttcgcctcgttatagc

SEQ ID NO: 246 - DNA, 20 bp, Artificial Sequence
  Synthetic oligonucleotide
gctataacgaggcgaacaca

SEQ ID NO: 247 - DNA, 20 bp, Artificial Sequence
  Synthetic oligonucleotide
ttaaggcggtctatcgaggt

SEQ ID NO: 248 - DNA, 20 bp, Artificial Sequence
  Synthetic oligonucleotide
acctcgatagaccgccttaa

SEQ ID NO: 249 - DNA, 20 bp, Artificial Sequence
  Synthetic oligonucleotide
ttagacgggttagttgcgat

SEQ ID NO: 250 - DNA, 20 bp, Artificial Sequence
  Synthetic oligonucleotide
atcgcaactaacccgtctaa

SEQ ID NO: 251 - DNA, 20 bp, Artificial Sequence
  Synthetic oligonucleotide
ttcattcgagtagcggttgg

SEQ ID NO: 252 - DNA, 20 bp, Artificial Sequence
  Synthetic oligonucleotide
ccaaccgctactcgaatgaa

SEQ ID NO: 253 - DNA, 20 bp, Artificial Sequence
  Synthetic oligonucleotide
ttcccgttagccgttatgtg

SEQ ID NO: 254 - DNA, 20 bp, Artificial Sequence
  Synthetic oligonucleotide
cacataacggctaacgggaa

SEQ ID NO: 255 - DNA, 20 bp, Artificial Sequence
  Synthetic oligonucleotide
ttctgattcagcgtggagtt

SEQ ID NO: 256 - DNA, 20 bp, Artificial Sequence
  Synthetic oligonucleotide
aactccacgctgaatcagaa

SEQ ID NO: 257 - DNA, 20 bp, Artificial Sequence
  Synthetic oligonucleotide
ttgagtgcttcgtattccgt

SEQ ID NO: 258 - DNA, 20 bp, Artificial Sequence
  Synthetic oligonucleotide
acggaatacgaagcactcaa

SEQ ID NO: 259 - DNA, 20 bp, Artificial Sequence
  Synthetic oligonucleotide
ttgatgctctcgggtgtatg

SEQ ID NO: 260 - DNA, 20 bp, Artificial Sequence
  Synthetic oligonucleotide
catacacccgagagcatcaa

SEQ ID NO: 261 - DNA, 20 bp, Artificial Sequence
  Synthetic oligonucleotide
ttgggtactctcgtagatgc

SEQ ID NO: 262 - DNA, 20 bp, Artificial Sequence
  Synthetic oligonucleotide
gcatctacgagagtacccaa

SEQ ID NO: 263 - DNA, 20 bp, Artificial Sequence
  Synthetic oligonucleotide
ttgtttcagggtattgggct

SEQ ID NO: 264 - DNA, 20 bp, Artificial Sequence
  Synthetic oligonucleotide
agcccaataccctgaaacaa

SEQ ID NO: 265 - DNA, 20 bp, Artificial Sequence
  Synthetic oligonucleotide
tttactcgtccgagcgtatg

SEQ ID NO: 266 - DNA, 20 bp, Artificial Sequence
  Synthetic oligonucleotide
catacgctcggacgagtaaa